(19) United States
(12) Patent Application Publication
(10) Pub. No.: US 2010/0256220 A1
(43) Pub. Date: Oct. 7, 2010

(54) PAS Kinase Regulates Energy Homeostasis

(75) Inventors: Jared Rutter, Salt Lake City, UT (US); Huai-Xiang Hao, Salt Lake City, UT (US); Wojtek Swiatek, Salt Lake City, UT (US)

Correspondence Address: Ballard Spahr LLP, SUITE 1000, 999 PEACHTREE STREET, ATLANTA, GA, 30309-3915, US

(73) Assignee: University Of Utah Research Foundation, Salt Lake City, UT (US)

(21) Appl. No.: 12/303,900

(22) PCT Filed: Jun. 7, 2007
(86) PCT No.: PCT/US07/70641

§ 371 Date: Mar. 4, 2009

Related U.S. Application Data
(60) Provisional application No. 60/811,819, filed on Jun. 8, 2006.

(51) Int. Cl. A61K 31/7088; A61P 3/10; A61P 3/04; C40B 30/00; C12Q 1/48

(52) U.S. Cl. 514/44 R; 506/7; 435/15

(57) ABSTRACT

Disclosed are compositions and methods related to PAS Kinase (PASK) and various diseases and disorders associated therewith. Included are methods for treating insulin resistance, cancer, and diabetes comprising administering a composition that inhibits PAS Kinase. Further included are methods, including high throughput screening methods, for identifying test compounds that modulate PAS Kinase.

### II. CROSS REFERENCE TO RELATED APPLICATIONS

This application claims the benefit of priority of U.S. Provisional Application No. 60/811,819, filed Jun. 8, 2006, which application is incorporated herein by this reference in its entirety.

### I. ACKNOWLEDGEMENTS

This invention was made with government support under NIH Grant RO1 DK071962. The government has certain rights in this invention.

### III. BACKGROUND

Due to changes in diet and lifestyle, the incidence of obesity and type 2 diabetes has been increasing dramatically worldwide. According to the U.S. National Institute of Health, around ⅔ of American adults are currently overweight or obese (Services 2001). Type 2 diabetes arises when pancreatic B cells fail to secrete sufficient insulin to compensate for peripheral insulin resistance, a condition severely aggravated by obesity (Rhodes 2005, Lazar 2005). Type 2 diabetes is now widely viewed as a manifestation of a broader underlying metabolic disorder called metabolic syndrome, which is characterized by hyperglycemia, hyperinsulinemia, dyslipidemia, hypertension, visceral obesity, and cardiovascular disease (Zimmet 2001). The World Health Organization estimates that the current decade will witness a 46% increase in diabetes incidence worldwide (from 151 million to 221 million), with the vast majority of this increase being due to metabolic syndrome-related Type 2 diabetes. What is needed in the art are methods and compositions for regulating PASK.

### IV. SUMMARY

Disclosed herein are methods of treating insulin resistance in a subject comprising selecting a subject in need of treatment for insulin resistance; and administering to the subject an effective amount of a composition that inhibits PASK.

Disclosed herein are methods of increasing mitochondrial metabolism in a cell, comprising inhibiting PASK, thereby increasing mitochondrial metabolism.

Also disclosed are methods of screening for a test compound that modulates PASK comprising contacting PASK with a test compound; and detecting interaction between PASK and the test compound; wherein interaction between the test compound and PASK indicates a compound that modulates PASK.

Disclosed is a method of screening for a test compound that modulates PASK comprising contacting a transgenic animal that is PASK deficient with a test compound; and detecting a difference in PASK levels in the transgenic animal; wherein a difference in PASK levels indicates a test compound that modulates PASK.

Disclosed herein is a method of treating cancer in a subject, comprising selecting a subject with cancer; and administering to the subject an effective amount of a composition that inhibits PASK; thereby treating cancer in the subject.

Also disclosed is a method of treating diabetes type I in a subject, comprising selecting a subject with diabetes type I; and administering to the subject an effective amount of a composition that inhibits PASK; thereby treating diabetes type I in the subject.

Also disclosed are methods of treating insulin resistance in a subject comprising: selecting a subject in need of treatment for insulin resistance; administering to the subject a nucleic acid encoding a composition that inhibits PASK.

### V. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an analysis of insulin secretion and sensitivity in PASK−/− mice. a, Plasma insulin concentrations before and after intraperitoneal glucose injection (n=24 male mice, 12 weeks of age). b, Perifusion of 15-17 isolated islets with a 3 mM to 30 mM glucose ramp in gassed Krebs buffer over 40 min (n=4 female mice, 16 weeks of age). Area under the curve: 1146±129 for WT islets and 646±171 for PASK−/− islets. The experiment was performed twice with similar results. *, P<0.05. c-f, Plasma glucose levels before and at indicated time points after intraperitoneal glucose (c, e) or insulin (d, f) injection. Data represent the average±s.e.m. for 12 male mice of each genotype, fed with either normal chow diet (c, d), or high fat diet (e, f) beginning from 12 week old. *, P<0.05, **, P<0.01.

FIG. 2 shows PASK−/− mice were protected from diet-induced obesity. a, b, Growth curves of wild-type and PASK−/− mice on normal chow diet (a) or high fat diet (b). Data represent the average±s.e.m. for 12 male mice of each genotype, fed with either NCD or HFD beginning from 12 week old. *, P<0.05, **, P<0.01. c, Representative Oil red 0 staining of liver cryosections from 24 week-old wild type and PASK−/− male mice on NCD and HFD following an 8 h fast. Oil Red O stains neutral lipid. d, Liver triglyceride content in 24 week-old wild type and PASK−/− mice on NCD and HFD under 8 h fasted conditions (n=3-5 male mice each genotype).

FIG. 3 shows PASK^{-/-} mice exhibit increased metabolic rate without increased mitochondrial mass. A, Metabolic chamber analysis of WT and PASK^{-/-} mice (n=3 male mice, 24 weeks of age). VO₂═O₂ consumption, VCO₂═CO₂ emission, and laser beam breaks are a measure of locomotor activity. For each parameter, the average of the WT value was set at 100%. Data presented are values from night (6 pm to 6 am). Daytime values exhibited a similar difference between WT and PASK^{-/-} mice. B, Maximal ATP production rate in saponin-permeabilized soleus muscle fibers, measured in the presence of 1 mM exogenous ADP and succinate (n=10 male mice, 16 weeks of age). C, Representative electron micrographs of soleus muscle from 16 week-old WT and PASK^{-/-} male mice. The EM image shown was at 8000× magnification. Four sections from each animal and 3 animals per genotype were examined. D, Quantification of mitochondrial number manually counted from 10 electron micrographs per genotype. Data presented is the average±SD and is normalized to Z line number. E, Citrate synthase activity in soleus muscle extracts from WT and PASK^{-/-} mice (n=6 male mice, 16 weeks of age). Data presented is the average±SD.

FIG. 4 shows AMPK expression and activity were up-regulated by PASK inactivation. a, The protein levels of AMPK, Phospho-AMPK(T172), ACC (acetyl-coA carboxylase) and Phospho-ACC(S79) in livers from WT and PASK−/− mice on NCD. b, The AMPK protein levels in gastrocnemius and soleus muscle and in isolated islets from NCD-fed WT and PASK−/− mice, c, AMPK protein levels in cultured HEK293 cells and rat L6 myocytes at 24 h and 48 h after infection with adenovirus expressing small hairpin RNA (shRNA) constructs targeting PASK. UI=uninfected cells, Scr=scrambled shRNA, #1=PASK shRNA-1, #2=PASK shRNA-2. d, AMPK protein levels in gastrocnemius muscle from age-matched WT and PASK−/− mice on NCD or HFD. Each lane in a-d represents 50 ug of protein sample.

FIG. 5 shows reduced triglyceride accumulation in PASK^{-/-} livers on HFD. A, Immunoblot analysis of Phospho-AMPK (Thr172), Phospho-S6K (Thr389) and tubulin (as loading control) levels in liver extracts from WT and PASK^{-/-} mice on NCD and HFD. Two mice per group are shown. Total AMPK and S6K protein levels were also analyzed and found to be identical among the groups. B, SCD1, FAE, CD36, PPARγ, CYP3A11 and PXR mRNA levels in liver from WT and PASK^{-/-} mice on HFD measured by qRT-PCR. Data shown are the average of n=6 per group with the WT value set as 1. RPL13A was used as the normalizer. All data presented is the average±SD.

FIG. 6 shows increased oxidative metabolism and cellular ATP content upon PASK silencing in L6 cells. A, PASK mRNA levels in L6 clones expressing either scrambled or PASK shRNA in the absence of doxycycline. Data represent the average±SD of triplicates per group. B, Measurement of ¹⁴CO₂ released from indicated L6 clones incubated in ¹⁴C-glucose (n=3). C, Measurement of ¹⁴CO₂ released from indicated L6 clones incubated in ¹⁴C-palmitate (n=3). D, Cellular ATP content measurement normalized to protein in extract.

FIG. 7 shows PASK expression is induced by refeeding in liver and HepG2 cells. A, qRT-PCR analysis of PASK and SREBP-1c mRNA levels in liver and adipose tissue from 19 hr fasted and 12 hr fasted/7 hr refed mice. Data represent the average±SD of 6 mice per group. B, qRT-PCR analysis of hPASK mRNA levels in HepG2 cells. 0G0S=starvation medium lacking glucose and serum, HG0S=refeeding medium with 30 mM glucose but no serum, 0GHS=refeeding medium with 15% serum but no glucose, HGHS=refeeding medium with 30 mM glucose and 15% serum. RNA was extracted from HepG2 cells that have been starved in 0G0S starvation medium for 24 hr, followed by 7 hr stimulation in the four indicated conditions. Data represent the average±SD of duplicates per group. C, Immunoblot analysis of hPASK and tubulin protein levels in HepG2 cells. HepG2 cells were treated the same except the duration in the indicated medium was increased to 24 hr. Cells in 6-well plates were directly lysed in 1×SDS-PAGE loading buffer and analyzed.

FIG. 8 shows PASK^{-/-} mice on HFD exhibited reduced circulating insulin levels and adiposity. A, Measurement of plasma insulin levels from 24 week-old HFD WT and PASK^{-/-} male mice fasted for 8 hr. Data represent the average±SD of 10 mice per genotype. B, Body fat mass in 23 week-old WT and PASK^{-/-} mice on HFD, measured by dual energy X-ray absorptiometry (DEXA). Data represent the average±SD of 6 mice per genotype.

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The term “multiwell plate” refers to a two dimensional array of addressable wells located on a substantially flat surface. Multiwell plates can include any number of discrete addressable wells, and include addressable wells of any width or depth. Common examples of multiwell plates include 96 well plates, 384 well plates and 3456 well Nanoplates™. Such multiwell plates can be constructed of plastic, glass, or any essentially electrically nonconductive material

The term “gene knockout” as used herein, refers to the targeted disruption of a gene in vivo with complete loss of function that has been achieved by any transgenic technology familiar to those in the art. In one embodiment, transgenic animals having gene knockouts are those in which the target gene has been rendered nonfunctional by an insertion targeted to the gene to be rendered non-functional by homologous recombination.

The term “hit” refers to a test compound that shows desired properties in an assay.

The term “repetitive” means to repeat at least twice.

The term “test compound” refers to a chemical to be tested by one or more screening method(s) of the invention as a putative modulator. A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar and 10 micromolar. Test compound controls can include the measurement of a signal in the absence of the test compound or comparison to a compound known to modulate the target.

The term “transgenic” is used to describe an organism that includes exogenous genetic material within all of its cells. The term includes any organism whose genome has been altered by in vitro manipulation of the early embryo or fertilized egg or by any transgenic technology to induce a specific gene knockout.

The term “transgene” refers to any piece of DNA which is inserted by artifice into a cell, and becomes part of the genome of the organism (i.e., either stably integrated or as a stable extrachromosomal element) which develops from that cell. Such a transgene can include a gene which is partly or entirely heterologous (i.e., foreign) to the transgenic organism, or may represent a gene homologous to an endogenous gene of the organism. Included within this definition is a transgene created by the providing of an RNA sequence that is transcribed into DNA and then incorporated into the genome. The transgenes of the invention include DNA sequences that encode the fluorescent or bioluminescent protein that may be expressed in a transgenic non-human animal.

The term “activity” as used herein refers to a measurable result of the interaction of molecules. Some exemplary methods of measuring these activities are provided herein.

The term “modulate” as used herein refers to the ability of a compound to change an activity in some measurable way as compared to an appropriate control. As a result of the presence of compounds in the assays, activities can increase (e.g. there could be increased levels of PASK or downstream genes/proteins of PASK), or “decrease” (e.g. there could be decreased levels of PASK or downstream genes/proteins of PASK) as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “monitoring” as used herein refers to any method in the art by which an activity can be measured.

The term “providing” as used herein refers to any means of adding a compound or molecule to something known in the art. Examples of providing can include the use of pipets, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

The term “treating” as used herein refers to administering a compound after the onset of clinical symptoms.

The term “in need of treatment” as used herein refers to a judgment made by a caregiver (e.g. physician, nurse, nurse practitioner, or individual in the case of humans; veterinarian in the case of animals, including non-human mammals) that an individual or animal requires or will benefit from treatment. This judgment is made based on a variety of factors that are in the realm of a care giver's expertise, but that include the knowledge that the individual or animal is ill, or will be ill, as the result of a condition that is treatable by the compounds of the invention.

The term “individual” as used herein refers to a mammal, including animals, preferably mice, rats, other rodents, rabbits, dogs, cats, swine, cattle, sheep, horses, or primates, most preferably humans.

The term “non-human animal” refers to any non-human vertebrate, birds and more usually mammals, preferably primates, animals such as swine, goats, sheep, donkeys, horses, cats, dogs, rabbits or rodents, more preferably rats or mice. Both the terms “animal” and “mammal” expressly embrace human subjects unless preceded with the term “non-human”.

The terms “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. GENERAL

Like AMPK, PAS kinase (PASK) is a nutrient-responsive protein kinase conserved from yeast to man. The PAS domain of PASK specifically interacts with the kinase catalytic domain and inactivates the kinase in cis (Rutter 2001). Based on biochemical and genetic data, it appears a small metabolite activates PASK by directly interacting with the PAS domain and disrupting its interaction with the kinase domain. Studies in cultured pancreatic B cells (da Silvia 2004) support a role for PASK in nutrient sensing and response. It was found that elevated medium glucose concentrations caused post-translational activation of PASK. It was also demonstrated that PASK activity is required for glucose-stimulated insulin expression (da Silvia 2004). However, the in vivo role of PASK in pancreatic B cell function and energy homeostasis was not previously addressed. Using PASK^{-/-} mice, it has been demonstrated that PASK is indeed required for normal insulin secretion by pancreatic B cells. It has also been demonstrated that PASK deletion results in nearly complete resistance to the phenotypes caused by a high-fat diet, including obesity, insulin resistance and hepatic fat accumulation. This protection is due to a striking increase in AMPK expression in each of the relevant tissues. Therefore, PASK inhibition can provide an effective therapeutic strategy for Type 2 diabetes, insulin resistance in general, and the metabolic syndrome.

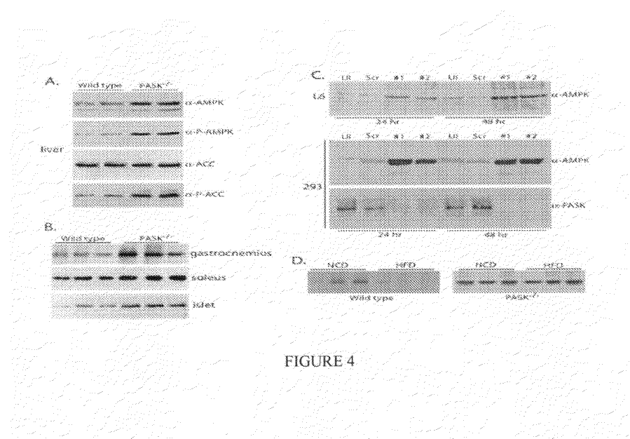

Therefore, described herein is a new therapeutic strategy for Type 2 Diabetes Mellitus, insulin resistance, and metabolic syndrome. Also described are methods for treating Type 1 Diabetes, and various forms of cancer. PASK deletion abrogates nearly all of the maladaptive phenotype associated with a high-fat diet, probably via maintenance of AMPK expression (see FIG. 4D). Increasing AMPK signaling is a proven therapeutic strategy, as illustrated by Metformin, which acts by increasing the phosphorylation and activation of AMPK. Inhibition of PASK signaling elicits similar beneficial effects, but through a completely distinct mechanism, namely increasing AMPK protein levels. This complementary therapeutic strategy, either alone or in combination, can be efficacious in the treatment of metabolic diseases.

Disclosed herein are methods of increasing AMPK production in a cell, comprising inhibiting PASK, thereby increasing AMPK. This can be done in vivo or in vitro. Also disclosed herein is a method of increasing mitochondrial metabolism a cell, comprising inhibiting PASK, thereby increasing mitochondrial metabolism. The subject in need of PASK inhibition can be identified prior to treatment.

Also disclosed are methods of treating insulin resistance in a subject comprising selecting a subject in need of treatment for insulin resistance; and administering to the subject an effective amount of a composition that inhibits PASK. Metabolic Syndrome (also known as Syndrome X) is characterized by having at least three of the following symptoms: insulin resistance; abdominal fat—in men this is defined as a 40 inch waist or larger, in women 35 inches or larger; high blood sugar levels—at least 110 milligrams per deciliter (mg/dL) after fasting; high triglycerides—at least 150 mg/dL in the blood stream; low HDL—less than 40 mg/dL; pro-thrombotic state (e.g. high fibrinogen or plasminogen activator inhibitor in the blood); or blood pressure of 130/85 mmHg or higher. A connection has been found between Metabolic Syndrome and other conditions such as obesity, high blood pressure and high levels of LDL “bad” cholesterol, all of which are risk factors for Cardiovascular Disease. For example, an increased link between Metabolic Syndrome and atherosclerosis has been shown. People with Metabolic Syndrome are also more prone to developing Type 2 Diabetes, as well as PCOS (Polycystic Ovarian Syndrome) in women and prostate cancer in men.

As described above, insulin resistance can be manifested in several ways, including Type 2 Diabetes. Type 2 diabetes is the condition most obviously linked to insulin resistance. Compensatory hyperinsulinemia helps maintain normal glucose levels—often for decades—before overt diabetes develops. Eventually the beta cells of the pancreas are unable to overcome insulin resistance through hypersecretion. Glucose levels rise, and a diagnosis of diabetes can be made. Patients with type 2 diabetes remain hyperinsulinemic until they are in an advanced stage of disease.

Insulin resistance can also include hypertension. One half of patients with essential hypertension are insulin resistant and hyperinsulinemic. There is evidence that blood pressure is linked to the degree of insulin resistance.

Hyperlipidemia is also associated with insulin resistance. The lipid profile of patients with type 2 diabetes includes decreased high-density lipoprotein cholesterol levels (a significant risk factor for heart disease), increased serum very-low-density lipoprotein cholesterol and triglyceride levels and, sometimes, a decreased low-density lipoprotein cholesterol level. Insulin resistance has been found in persons with low levels of high-density lipoprotein. Insulin levels have also been linked to very-low-density lipoprotein synthesis and plasma triglyceride.¹⁰

Atherosclerotic heart disease is also associated with insulin resistance, as is obesity. Many persons with one or more of the conditions listed above are obese. Obesity is a component of the syndrome, but it promotes insulin resistance rather than resulting from it.

Other abnormalities linked to insulin resistance include hyperuricemia, elevated levels of plasminogen activator inhibitor 1 and a preponderance of small-size, low-density lipoprotein particles. Higher plasminogen activator inhibitor 1 levels and decreased low-density lipoprotein particle diameter are thought to increase the risk of coronary heart disease.

Also disclosed are methods of screening for a test compound that modulates PASK. These steps include contacting PASK with a test compound; and detecting interaction between PASK and the test compound; wherein interaction between the test compound and PASK indicates a compound that modulates PASK. Methods of screening are further described below, and can include a high throughput assay system, such as an immobilized array of test compounds or PASK molecules. Also disclosed are compounds identified by the screening methods disclosed herein.

Also disclosed are methods of screening for a test compound that modulates PASK, wherein PASK is contacted with a test compound; and inhibition of PASK is monitored, thereby identifying a compound that modulates PASK. Methods of screening are further described below, and can include a high throughput assay system, such as an immobilized array of test compounds or PASK molecules. Also disclosed are compounds identified by the screening methods disclosed herein.

Also disclosed are in vivo screening methods. Disclosed is a method of screening for a test compound that modulates PASK comprising contacting a transgenic animal that is PASK deficient with a test compound; and detecting a difference in PASK levels in the transgenic animal; wherein a difference in PASK levels indicates a test compound that modulates PASK. Also disclosed are compounds identified by the in vivo screening methods disclosed herein.

Also disclosed are methods of treating cancer in a subject, comprising selecting a subject with cancer; and administering to the subject an effective amount of a composition that inhibits PASK; thereby treating cancer in the subject. Methods of treating cancer, as well as various cancers contemplated for use with these methods, are disclosed below.

Also disclosed are methods of treating Type I Diabetes in a subject, comprising selecting a subject with Type I Diabetes; and administering to the subject an effective amount of a composition that inhibits PASK; thereby treating diabetes type I in the subject.

Also disclosed are methods of treating insulin resistance in a subject comprising: selecting a subject in need of treatment for insulin resistance; and administering to the subject a nucleic acid encoding a composition that inhibits PASK. Methods of nucleic acid delivery are disclosed below.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Homology/Identity

It is understood that one way to define any known variants and derivatives or those that might arise, of the disclosed genes and proteins herein is through defining the variants and derivatives in terms of homology to specific known sequences. For example SEQ ID NO: 5 sets forth a particular sequence encoding a PASK molecule, and SEQ ID NO: 4 sets forth a particular sequence of the protein encoded by SEQ ID NO: 5, the PASK molecule. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

2. Nucleic acids

There are a variety of molecules disclosed herein that are nucleic acid based, including for example the nucleic acids that encode, for example, PASK as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, PASK as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of PASK or the genomic DNA of PASK or they can interact with the polypeptide PASK. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide.

Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

3. Nucleic Acid Delivery

In the methods described herein which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

4. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as a PASK inhibitor, into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase al transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pal, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wicicham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B 19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpes virus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed nucleic acids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Feigner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

5. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

6. Peptides

a) Protein Variants

As discussed herein there are numerous variants of the PASK proteins that are known and herein contemplated. In addition, to the known functional PASK strain variants there are derivatives of the PASK proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | Ala (A) |
| Allosoleucine | AIle |
| Arginine | Arg (R) |
| Asparagines | Asn (N) |
| aspartic acid | Asp (D) |
| Cysteine | Cys (C) |
| glutamic acid | Glu (E) |
| Glutamine | Gln (K) |
| Glycine | Gly (G) |
| Histidine | His (H) |
| Isolelucine | Ile (I) |
| Leucine | Leu (L) |
| Lysine | Lys (K) |
| Phenylalanine | Phe (F) |
| Praline | Pro (P) |
| pyroglutamic acid | PGlu |
| Serine | Ser (S} |
| Threonine | Thr (T) |
| Tyrosine | Tyr (Y) |
| Tryptophan | Trp (W) |
| Valine | Val (V) |

TABLE 2
Amino Acid Substitutions
Original Residue Exemplary Conservative
Substitutions, others areknown in the art.

Ala; ser
Arg; lys, gln
Asn; gln; his
Asp; glu
Cys; ser
Gln; asn, lys
Glu; asp
Gly; pro
His; asn; gln
Ile; leu; val
Leu; ile; val
Lys; arg; gln
Met; leu; ile
Phe; met; leu; tyr
Ser; thr
Thr; ser
Trp; tyr
Tyr; trp; phe
Val; ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 5 sets forth a particular sequence of PASK and SEQ ID NO: 4 sets forth a particular sequence of a PASK protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

7. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with PASK such that PASK is inhibited. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boemer et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti PASK antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

8. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their-formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing insulin resistance, cancer, or other diseases or disorders, the efficacy of the therapeutic antibody can be assessed in various ways well known to the skilled practitioner.

The compositions that inhibits PASK, or reduces PASK production, disclosed herein may be administered prophylactically to patients or subjects who are at risk for insulin resistance, or metabolic syndrome. Other molecules that interact with PASK but which do not have a specific pharmaceutical function, but which may be used for tracking changes within cellular chromosomes or for the delivery of diagnostic tools for example can be delivered in ways similar to those described for the pharmaceutical products.

The disclosed compositions and methods can also be used for example as tools to isolate and test new drug candidates for a variety of insulin and metabolic-related diseases.

9. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

10. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

11. Compositions Identified by Screening with Disclosed Compositions/Combinatorial Chemistry

a) Combinatorial Chemistry

The disclosed compositions can be used as targets for any combinatorial technique to identify molecules or macromolecular molecules that interact with the disclosed compositions in a desired way. Also disclosed are the compositions that are identified through combinatorial techniques or screening techniques in which the compositions disclosed herein, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, those that interact with PASK, are also disclosed. Thus, the products produced using the combinatorial or screening approaches that involve the disclosed compositions, such as PASK, are also considered herein disclosed.

It is understood that the disclosed methods for identifying molecules that inhibit PASK, for example, can be performed using high through put means. For example, putative inhibitors can be identified using Fluorescence Resonance Energy Transfer (FRET) to quickly identify interactions. The underlying theory of the techniques is that when two molecules are close in space, i.e., interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. No. 6,031,071; U.S. Pat. No. 5,824,520; U.S. Pat. No. 5,596,079; and U.S. Pat. No. 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example an extracellular portion of PASK is attached to a DNA binding domain of a transcriptional activation protein, such as Gal 4. By performing the Two-hybrid technique on this type of system, molecules that bind the extracellular portion of PASK can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. No. 6,017,768 and U.S. Pat. No. 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. No. 5,698,685 and U.S. Pat. No. 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in iterative processes.

12. Screening Methods

Disclosed herein is a method of screening for a test compound that modulates PASK comprising: contacting PASK with a test compound; and detecting interaction between PASK and the test compound; wherein interaction between the test compound and PASK indicates a compound that modulates PASK.

Also disclosed is a method of screening for a test compound that modulates PASK comprising: contacting a transgenic animal that is PASK deficient with a test compound; and detecting a difference in PASK levels in the transgenic animal; wherein a difference in PASK levels indicates a test compound that modulates PASK.

The modulation can comprise an increase in PASK or downstream activity. By an “increase” is meant that the activity is greater in the presence of the test compound than not in the presence of the test compound. The modulation can also comprise a decrease in PASK or downstream activity. By a “decrease” is meant that the activity is less in the presence of the test compound than not in the presence of the test compound.

The response of AMPK or downstream activity can be measured in the presence of various concentrations of test compound. The measuring steps can alsp comprise measuring the response at various concentrations of the test compound. For example, the concentration of the test compound can range from 1 nM to 1000 μM.

Assays contemplated by the invention include both binding assays and activity assays; these assays may be performed in conventional or high throughput formats. Modulator screens are designed to identify stimulatory and inhibitory agents. The sources for potential agents to be screened include natural sources, such as a cell extract (e.g., invertebrate cells including, but not limited to, bacterial, fungal, algal, and plant cells) and synthetic sources, such as chemical compound libraries or biological libraries such as antibody substance or peptide libraries. Agents are screened for the ability to either stimulate or inhibit the activity. Binding assays are used to detect activity levels. Both functional and binding assays of activity are readily adapted to screens for modulators such as agonist (stimulatory) and antagonist (inhibitory) compounds.

Contemplated herein are a multitude of assays to screen and identify modulators, such as agonists and antagonists, of PASK activity (and downstream activity). In one example, the PASK molecules are immobilized and interaction with a a candidate modulator is detected. In another example, the test compound is immobilized and the PASK is solubilized. In yet another example, interaction between PASK and the test compound is assessed in a solution assay. Another contemplated assay involves a variation of the di-hybrid assay wherein a modulator of protein/protein interactions is identified by detection of a positive signal in a transformed or transfected host cell.

Candidate modulators for screening according to contemplated by the invention include any chemical compounds, including libraries of chemical compounds. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, or analogs of known compounds, or analogs of compounds that have been identified as “hits” or “leads” in prior drug discovery screens, some of which may be derived from natural products or from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Candidate modulators contemplated by the invention can be designed and include soluble forms of binding partners, as well as chimeric, or fusion, proteins thereof. A “binding partner” as used herein broadly encompasses non-peptide modulators, peptide modulators (e.g., neuropeptide variants), antibodies (including monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR and/or antigen-binding sequences, which specifically recognize a polypeptide of the invention), antibody fragments, and modified compounds comprising antibody domains that are immunospecific for the expression product.

Assays that measure binding or interaction of compounds with target proteins include assays that identify compounds that inhibit unfolding or denaturation of a target protein, assays that separate compounds that bind to target proteins through, affinity ultrafiltration followed by ion spray mass spectroscopy/HPLC methods or other physical and analytical methods, capillary electrophoresis assays and two-hybrid assays.

One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene.

### a) Antibodies to Receptors as Modulators of Binding

Standard techniques are employed to generate polyclonal or monoclonal antibodies to receptors, and to generate useful antigen-binding fragments thereof or variants thereof. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988). In one embodiment, recombinant polypeptides (or cells or cell membranes containing such polypeptides) are used as antigens to generate the antibodies. In another embodiment, one or more peptides having amino acid sequences corresponding to an immunogenic portion of a receptor are used as antigen. Peptides corresponding to extracellular portions of receptors, especially hydrophilic extracellular portions, are preferred. The antigen may be mixed with an adjuvant or linked to a hapten to increase antibody production. Polyclonal and monoclonal antibodies, chimeric (e.g., humanized) antibodies, fragments of antibodies, and all other forms of antibody molecules disclosed herein are referred to collectively as antibody products.

### (1) Polyclonal or Monoclonal Antibodies

As one exemplary protocol, a recombinant polypeptide or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanin (Pierce), according to the manufacturer's recommendations. For an initial injection, the antigen is emulsified with Freund's Complete Adjuvant and injected subcutaneously. At intervals of two to three weeks, additional aliquots of receptor antigen are emulsified with Freund's Incomplete Adjuvant and injected subcutaneously. Prior to the final booster injection, a serum sample is taken from the immunized mice and assayed by Western blot to confirm the presence of antibodies that immunoreact with a polypeptide. Serum from the immunized animals may be used as a polyclonal antisera or used to isolate polyclonal antibodies that recognize a receptor. Alternatively, the mice are sacrificed and their spleens are removed for generation of monoclonal antibodies.

One example of generating monoclonal antibodies follows: the spleens are placed in 10 ml serum-free RPMI 1640, and single-cell suspensions are formed by grinding the spleens in serum-free RPMI 1640, supplemented with 2 mM L-glutamine, 1 mM sodium pyruvate, 100 Units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% (FBS(Hyclone Laboratories, Inc., Logan, Utah) for three days prior to fusion, are centrifuged and washed as well.

One example of producing hybridoma fusions follows: spleen cells from the immunized mice can be combined with NS-1 cells and centrifuged, and the supernatant is aspirated. The cell pellet is dislodged by tapping the tube, and 2 ml of 37° C. PEG 1500 (50% in 75 mM HEPES, pH 8.0) (Boehringer-Mannheim) is stirred into the pellet, followed by the addition of serum-free RPMI. Thereafter, the cells are centrifuged and resuspended in RPMI containing 15% FBS, 100 .mu.M sodium hypoxanthine, 0.4 μM aminopterin, 16 μM thymidine (HAT) (Gibco), 25 Units/ml IL-6 (Boehringer-Mannheim) and 1.5×106 thymocytes/ml and plated into 10 Corning flat-bottom 96-well tissue culture plates (Corning, Corning N.Y.).

On days 2, 4, and 6 after the fusion, 100 μl of medium is removed from the wells of the fusion plates and replaced with fresh medium. On day 8, the fusions are screened by ELISA, testing for the presence of mouse IgG that binds to a receptor polypeptide. Selected fusions are further cloned by dilution until monoclonal cultures producing anti-receptor antibodies are obtained.

(2) Receptor-Neutralizing Antibodies from Phage Display

Receptor-neutralizing antibodies are generated by phage display techniques such as those described in Aujame et al., Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is screened for target-specific phage-antibodies using a labeled or immobilized target peptide or polypeptide as antigen-probe.

(3) Receptor-Neutralizing Antibodies from Transgenic Animals

Receptor-neutralizing antibodies are generated in transgenic animals, such as mice, essentially as described in Bruggemann et al., Immunol. Today 17(8):391-97 (1996) and Bruggemann et al., Curr. Opin. Biotechnol. 8:455-58 (1997). Transgenic mice carrying V-gene segments in germline configuration, and expressing the transgenes in their lymphoid tissue, are immunized with a polypeptide composition using conventional immunization protocols. Hybridomas are generated from B cells of the immunized mice using conventional protocols and screened to identify hybridomas secreting anti-receptor antibodies (e.g., as described above).

(4) High Throughput Screening (HTS) Systems for Drug Discovery

The literature is replete with examples of the use of radiolabeled ligands in HTS binding assays for drug discovery (see Williams, Med. Res. Rev. 11:147-184 (1991); Sweetnam et al., J. Nat. Prod. 56:441-455 (1993) herein incorporated by reference in their entirety for their teaching concerning high throughput screens). It is also possible to screen for novel neuroregeneration compounds with radiolabeled ligands in HTS binding screens. Other reasons that recombinant receptors are preferred for HTS binding assays include better specificity (higher relative purity) and ability to generate large amounts of receptor material (see Hodgson, Bio/Technology 10:973-980 (1992)).

A variety of heterologous systems are available for expression of recombinant proteins and are well known to those skilled in the art. Such systems include bacteria (Strosberg et al., Trends in Pharm. Sci. 13:95-98 (1992)), yeast (Pausch, Trends in Biotech. 15:487-494 (1997)), several kinds of insect cells (Vanden Broeck, Intl. Rev. Cytol. 164:189-268 (1996)), amphibian cells (Jayawickreme et al., Curr. Opin. Biotechnol. 8:629-634 (1997)) and several mammalian cell lines (CHO, HEK293, COS, etc.; see Gerhardt et al., Eur. J. Pharmacol. 334:1-23 (1997); Wilson et al., Brit. J. Pharmacol. 125:1387-1392 (1998)). These examples do not preclude the use of other possible cell expression systems, including cell lines obtained from nematodes (WO 98/37177).

### (5) Response-Based Receptor HTS Systems

Inhibition of PASK, or downstream products or genes of PASK, can result in a variety of biological responses, which are typically mediated by proteins expressed in the host cells. The proteins can be native constituents of the host cell or can be introduced through well-known recombinant technology. They can be mutants of native varieties as well. The proteins can be intact or chimeric.

Fluorescence changes can also be used to monitor ligand-induced changes in membrane potential or intracellular pH; an automated system suitable for HTS has been described for these purposes (Schroeder et al., J. Biomol. Screening 1:75-80 (1996)). Among the modulators that can be identified by these assays are natural ligand compounds; synthetic analogs and derivatives of natural ligands; antibodies, antibody fragments, and/or antibody-like compounds derived from natural antibodies or from antibody-like combinatorial libraries; and/or synthetic compounds identified by high throughput screening of libraries; and other libraries known in the art. All modulators that bind PASK are useful for identifying PASK-like polypeptides in tissue samples (e.g., for diagnostic purposes, pathological purposes, and other purposes known in the art). Agonist and antagonist modulators are useful for up-regulating and down-regulating PASK activity, respectively, for purposes described herein.

The assays may be performed using single putative modulators; they may also be performed using a known agonist in combination with candidate antagonists (or visa versa). Detectable molecules that may be used include, but are not limited to, molecules that are detectable by spectroscopic, photochemical, biochemical, immunochemical, electrical, radioactive, and optical means, including but not limited to bioluminescence, phosphorescence, and fluorescence. These detectable molecules should be a biologically compatible molecule and should not compromise the biological function of the molecule and must not compromise the ability of the detectable molecule to be detected. Preferred detectable molecules are optically detectable molecules, including optically detectable proteins, such that they may be excited chemically, mechanically, electrically, or radioactively to emit fluorescence, phosphorescence, or bioluminescence. More preferred detectable molecules are inherently fluorescent molecules, such as fluorescent proteins, including, for example, Green Fluorescent Protein (GFP). The detectable molecule may be conjugated to the GRK protein by methods as described in Barak et al. (U.S. Pat. Nos. 5,891,646 and 6,110,693). The detectable molecule may be conjugated at the front-end, at the back-end, or in the middle.

b) Nucleic Acid Detection

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2.mu. plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, plant cells, nematode cells, and animal cells, such as HEK-293, CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

c) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, inhibitors of PASK, are also disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

13. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, disclosed is a kit for treating insulin resistance in a subject, comprising the compositions disclosed herein.

### D. METHODS OF USING THE COMPOSITIONS

1. Methods of Using the Compositions as Research Tools

The disclosed compositions can be used in a variety of ways as research tools. For example, the disclosed compositions can be used to study the relationship between PASK and its downstream genes by for example acting as inhibitors of binding.

2. Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination events occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

3. Method of Treating Cancer

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### PAS Kinase Regulates Energy Homeostasis Through Inhibition of AMPK Expression

a) Analysis of Insulin Secretion and Action in PASK^{-/-} Mice

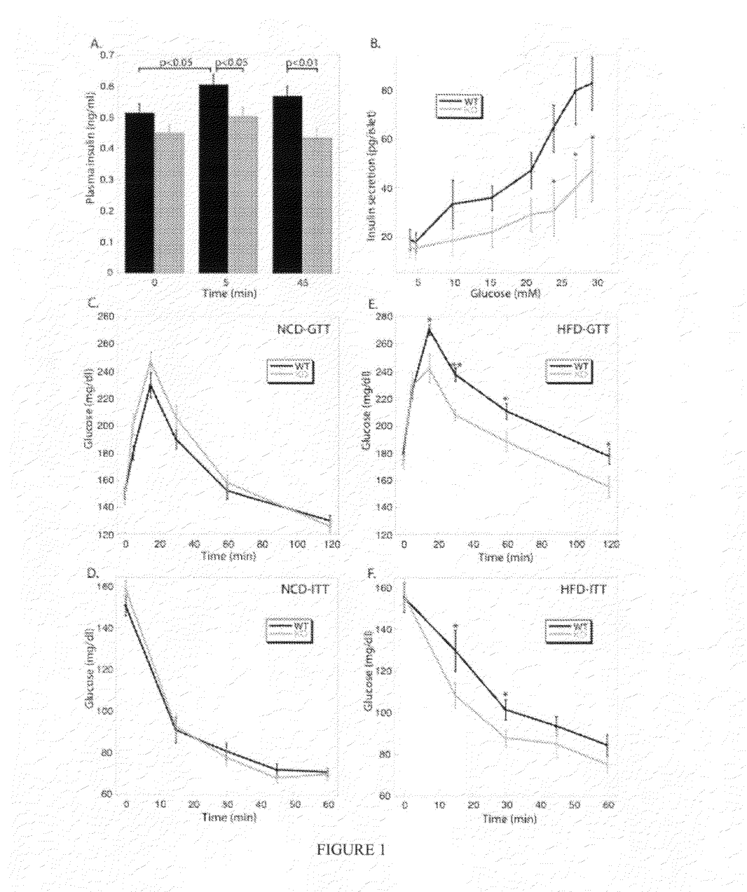

To examine glucose-stimulated insulin secretion (GSIS) in PASK^{-/-} mice, the plasma insulin levels were measured before and after an intraperitoneal glucose injection. As shown in FIG. 1a, insulin levels in PASK^{-/-} mice are significantly lower than wild-type (WT) littermates at both 5 min and 45 min following glucose injection. This insulin secretion defect is also manifest in isolated islets of Langerhans in vitro. Using islet perifusion experiment, PASK^{-/-} islets were shown to be defective in GSIS, especially at high glucose concentrations (FIG. 1b). The total insulin secreted by PASK^{-/-} islets in these experiments was only 56% of that of WT islets.

To assess whether this hypoinsulinemia resulted in impaired overall glucose homeostasis, a glucose tolerance test (GTT) was performed, in which plasma glucose levels were monitored over time following glucose injection. PASK^{-/-} mice displayed slight but not statistically significant glucose intolerance (FIG. 1c), consistent with the modest hypoinsulinemic phenotype. Insulin sensitivity, as measured by insulin tolerance test (ITT), was not obviously different between WT and PASK^{-/-} mice (FIG. 1d).

When fed a high-fat diet (H D), C57BL/6J mice are known to develop obesity and a set of symptoms reminiscent of the metabolic syndrome, including insulin resistance (Surwit 1988). To examine pancreatic β cell function of PASK^{-/-} mice under conditions of increased insulin demand, they were fed a HFD from 12 week old and performed GTT and ITT experiments. HFD-fed PASK^{-/-} mice showed better glucose tolerance (FIG. 1e) and increased insulin sensitivity (FIG. 1f) relative to WT littermates. In fact, HFD-feeding causes defects in glucose tolerance and insulin sensitivity in WT mice, but has little or no effect in PAN& mice (FIG. 1c versus FIG. 1e, FIG. 1d versus FIG. 10. As expected from the increased insulin sensitivity, fasting insulin levels of HFD-fed PASK^{-/-} mice were less than half that of WT mice.

b) PASK^{-/-} Mice were Protected from Diet-Induced Obesity.

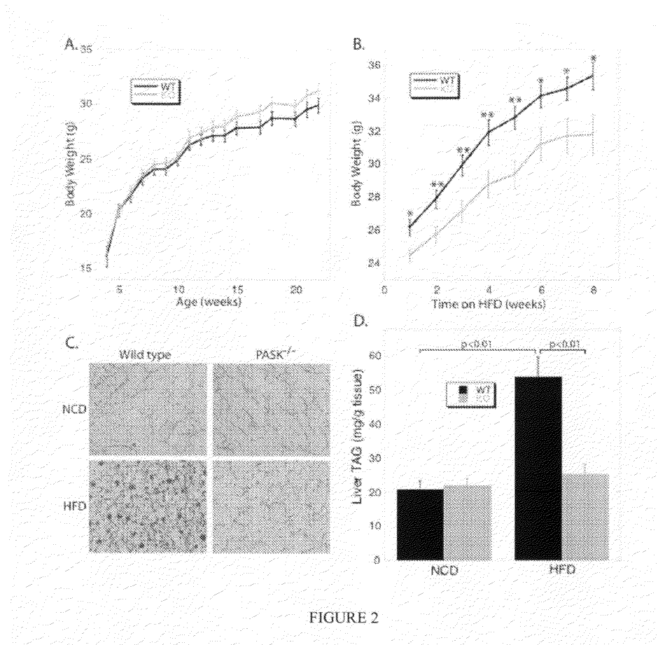

In addition to the protection from insulin resistance, PASK^{-/-} mice were also protected from HFD-induced obesity. WT and PASK^{-/-} mice gained similar amount of weight on NCD (FIG. 2a), but the weight gain of PASK^{-/-} mice on HFD was significantly less than that of WT littermates (FIG. 2b). After 8 weeks of HFD-feeding, the body weight of PASK^{-/-} mice was similar to age-matched NCD-fed PASK^{-/-} mice. Analysis of body composition by dual energy X-ray absorptiometry confirmed that the difference in total body weight is completely due to decreased fat mass in PASK^{-/-} mice.

Diet-induced obesity is typically accompanied by increased triglyceride accumulation in peripheral tissues, which is strongly associated with the development of insulin resistance (Voshol 2003). The liver triglyceride content was examined both by histological staining (FIG. 2c) and by enzymatic quantification (FIG. 2d). In both cases, PASK^{-/-} mice were shown to be completely protected from the increased lipid accumulation in liver observed in WT mice on HFD versus NCD. Indeed, liver triglyceride levels of HFD-fed PASK^{-/-} mice were nearly indistinguishable from those of NCD-fed WT or PASK^{-/-} mice.

c) PASK^{-/-} Mice Had an Increased Metabolic Rate.

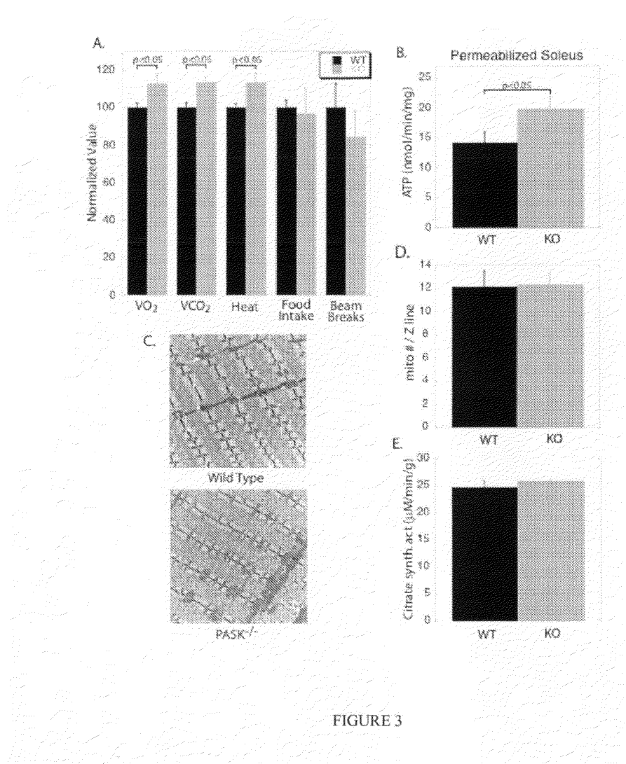

Obesity is the result of imbalance between energy intake (in the form of feeding) and energy expenditure (in the form of physical activity and basal metabolism) (Spiegelman 2001). To determine the mechanism of the lean phenotype in PASK^{-/-} mice, food intake, locomotor activity, O₂ consumption and CO₂ emission were measured by indirect calorimetry using metabolic chambers. The food intake and locomotor activity were similar in PASK^{-/-} mice and WT littermates, but PASK^{-/-} mice consumed more O₂, emitted more CO₂ and generated more heat both during the day and the night (FIG. 3a). This hypermetabolic phenotype in PASK^{-/-} mice was also manifest in permeabilized soleus muscle fibers, wherein the maximal ATP production rate was higher in PASK^{-/-} muscle relative to WT muscle (FIG. 3b).

One explanation for the observed hypermetabolic phenotype and increased ATP synthesis was an increase in mitochondrial number or mass in PASK^{-/-} muscle. However, soleus muscle electron micrographs (FIG. 3c) and subsequent quantification showed no significant increase of mitochondrial number/mass between PASK^{-/-} and WT soleus muscle. Further, the activity of citrate synthase, a marker of mitochondrial density (Leek 2001), was identical in PASK^{-/-} and WT soleus muscle extracts (FIG. 3d).

d) AMPK Expression and Activity were Up-Regulated in PASK^{-/-} Tissues.

AMPK expression and activity were explored in PASK^{-/-} mice. As shown in FIG. 4a, AMPK protein level was markedly elevated in liver extracts from PASK^{-/-} mice. This change in total protein level appears to result in increased AMPK signaling. A robust increase in the phosphorylated or active form of AMPK was observed. Also observed was increased phosphorylation of acetyl-coA carboxylase (ACC), a known direct phosphorylation target of AMPK (Shaw, 2004). Further, we observed an increase in the known AMPK-responsive mRNAs.

AMPK levels were examined in gastrocnemius and soleus skeletal muscle and in pancreatic islets. In all three cases, PASK^{-/-} tissue exhibited increased AMPK protein levels (FIG. 4b). To address the possibility that the observed increase in AMPK is a secondary or physiological adaptive response to PASK deletion, the effects of acute knock-down of PASK in cultured cells was examined. Both in HEK293 cells and L6 myocytes, PASK suppression resulted in robust increases in AMPK expression (FIG. 4c). As recapitulation of the increase in AMPK levels in cultured cells under constant medium conditions occurred, it was concluded that it is not a secondary effect due to altered hormone or nutrient conditions in PASK^{-/-} mice.

It has been previously demonstrated that maintenance on a HFD results in decreased AMPK expression in skeletal muscle (Liu, 2006). This loss of AMPK was suggested to be important in the pathogenesis of the metabolic dysfunction associated with this dietary regime. To address a potential role of PASK in mediating these effects, AMPK levels were examined in gastrocnemius muscle of WT and PASK^{-/-} mice maintained on either a NCD or HFD. A decrease in AMPK levels in HFD-fed WT mice relative to those fed a NCD was observed. This decrease, however, was completely absent in PASK^{-/-} mice. Therefore, PASK is required for the loss of AMPK observed in HFD-fed skeletal muscle.

e) Discussion

Herein, a series of tissue-specific metabolic phenotypes associated with deletion of the PASK gene in mice have been described. Increased AMPK protein levels have also been observed, which is likely responsible for the phenotype in each of these tissues. AMPK over-expression has been associated with decreased insulin production and secretion in pancreatic n-cells in vitro and in vivo (da Silva 2003, Richards 2005). Hepatic lipid accumulation has also been shown to be ameliorated with AMPK activation (Winder 1999). Finally, AMPK activation has been implicated in increasing oxidative metabolism in skeletal muscle (Hardie 2003). Therefore, each of the cellular and tissue-specific phenotypes described are due to activation of cellular AMPK signaling.

The PASK-AMPK interaction represents a clear example of cross-talk between cellular metabolic sensing pathways. PASK is activated by stimulatory glucose concentrations in pancreatic β cells, and PASK acts as a cell-autonomous nutrient or metabolic sufficiency sensor.

f) Methods

Animals. PASK^{-/-} mice were genotyped as described (Katschinski 2003).

After the 5th backcross into C57BL/6J (The Jackson Laboratory), 12 week˜24 week old males were used for all experiments except the islet perifusion studies were performed on females. Mice were maintained on a normal chow diet (Harlan Teklad 3080) or a high fat diet from 12 weeks of age (45% fat by calories, Research Diets, D12451). In each experiment, age-matched wild type littermates were used as controls for PASK^{-/-} mice.

Glucose/Insulin tolerance test and insulin secretion in vivo. For GTT and plasma insulin measurement, experimental animals were fasted for 6 h, after which glucose (1 g/kg body weight) was injected intraperitoneally. At the indicated times, tail vein blood was sampled for glucose determination with a glucometer (Bayer Corp.) or for insulin measurement using Sensitive Rat Insulin RIA Kit (Linco Research). For ITT, human recombinant insulin (Novo Nordisk, 0.75 U/kg body weight) was intraperitoneally administrated to random-fed mice and blood glucose levels were determined at the indicated times.

Islet isolation and perifusion. Islets of Langerhans were isolated from pancreas using the intraductal liberase (Roche) digestion method as described previously (Cooksey 2004). 15-17 size-matched islets were individually handpicked and used for perifusion experiments as described. The glucose and released insulin in fractions of perifusion buffer were measured using a Glucose Analyzer (Beckman Instruments) and Sensitive Rat Insulin RIA Kit (Linco Research), respectively.

Liver triglyceride content measurement. Quantitative analysis of liver triglyceride content was performed by saponification of liver in ethanolic KOH as described (Norris 2003). After neutralization with MgCl₂, glycerol produced during hydrolysis was measured by a colorimetric assay using Free Glycerol Reagent and Glycerol Standard Solution (Sigma).

Histology and electron microscopy. For Oil Red O staining, frozen liver samples were embedded in OTC reagent (Tissue-Tek) and sectioned at 8 um in a cryostat. Cryosections were fixed in formaldehyde steam at 50° C., incubated in 0.5% Oil Red O in isopropyl alcohol and counter-stained with hematoxylin (Sigma). For EM, 3 pairs of WT and PASK^{-/-} soleus muscle were fixed, dehydrated in graded ethanol and embedded in Poly Bed plastic resins for sectioning as described (Leone 2005). Soleus mitochondrial number were quantified in blinded fashion from electron micrographs at the magnification of 8000× and normalized to Z line number.

Metabolic chamber studies. Indirect calorimetry was performed with a four-chamber Oxymax system (Columbus Instruments). Animals were allowed to adapt to the metabolic chamber for 4 hours and then VO₂, VCO₂, body heat, food and water intake, and movement were measured every 15 minutes for 3 days from individually-housed mice. Averaged data from 6 pm to 6 am is expressed as night values while data from 6 am to 6 pm is expressed as day values.

Mitochondrial respiration experiment. Soleus fibers were separated and then permeabilized by saponin. Maximal (ADP-stimulated) ATP production rate was determined by exposing fibers to 1 mM exogenous ADP as described (Leone 2005). Succinate was used as substrate.

Citrate synthase activity assay. The CS activity was measured by a spectrophotometric method as described (Boudina 2005). Briefly, frozen soleus muscle was homogenized on ice and citrate synthase were released from mitochondria by freezing and thawing the homogenate. The reaction was initiated by addition of oxaloacetate into 1 ml diluted homogenate in reaction buffer containing acetyl-CoA, and then monitored at 412 nm for 3 minutes with an Ultrospec 3000 spectrophotometer (Amersham).

RNA interference and adenovirus generation. shRNA oligonucleotide duplexes containing sequences targeting human, mouse and rat PASK gene and a scrambled shRNA were designed using Invitrogen website software and cloned into adenoviral shRNA vector pAd shRNA/hU6. Adenoviruses were produced from these constructs using the AdEasy™ Adenoviral Vector System (Stratagene), according to the manufacturer's instruction. shRNA sequences: PASK#1-GATGCCAAGACCACAGAGA (SEQ ID NO: 1), PASK#2-GCGCAGACAAGCTCAAAGA (SEQ ID NO: 2), and Scrambled-GCGCAGACAAGCTCAAAGA (SEQ ID NO: 3).

Western blot. Lysate of tissues (isolated islets, gastrocnemius and soleus muscle, and liver) or cells (HEK 293 cells, rat L6 myocytes) were prepared using lysis buffer after homogenization and sonication where applicable. About 50 ug of proteins from each sample, determined by Advanced Protein Assay Reagent (Cytoskeleton, Inc), were separated by SDS-PAGE, transferred to a PVDF membrane (Fisher), and blotted with indicated antibodies following the manufacturer's protocol. AMPK, Phospho-AMPK(T172), ACC and Phospho-ACC(S79) were purchased from Cell Signalling Inc.

Real-time quantitative RT-PCR. Total RNA was extracted from about 100 mg liver samples using RNAStat60 reagent (Tel-Test Inc.) and purified with RNeasy Mini Kit (Qiagen), according to the manufacturer's instructions. First-strand cDNA synthesis was carried out using Superscript III reverse transcriptase Kit (Invitrogen). Real-time PCR was performed on Roche LightCycler using the SYBR Green-based method as described (Cooksey 2004). Melting curve analysis and a mock reverse transcribed control were included to ensure the specificity of the amplicon. Primer sequences for indicated transcripts are available upon request.

Statistical analysis. Data are presented as average±s.e.m. A two-tailed equal variance t-test was used to compare differences between WT and PASK^{-/-} littermates, and the null hypothesis was rejected at the 0.05 level.

### Example 2

### PAS Kinase is Required for Normal Cellular Energy Balance

a) Summary

The metabolic syndrome, a complex set of phenotypes typically associated with obesity and diabetes, is an increasing threat to global public health. Fundamentally, the metabolic syndrome is caused by a failure to properly sense and respond to cellular metabolic cues. The role of the cellular metabolic sensor, PAS kinase (PASK), was studied in the pathogenesis of metabolic disease using PASK^{-/-} mice. Tissue-specific metabolic phenotypes caused by PASK deletion were found consistent with its role as a metabolic sensor. Specifically, PASK^{-/-} mice exhibited altered triglyceride storage in liver and increased metabolic rate in skeletal muscle. Further, PASK deletion caused nearly complete protection from the deleterious effects of a high fat diet including obesity and insulin resistance. Consistent with its role in metabolic sensing, PASK mRNA in liver is acutely induced by refeeding. It was also demonstrated that these effects, i.e., increased rate of oxidative metabolism and feeding regulation of PASK mRNA, occur in cultured cells. It therefore appears that PASK acts in a cell-autonomous manner to maintain cellular energy homeostasis and is a potential therapeutic target for metabolic disease.

b) Introduction

Due to changes in diet and lifestyle, the incidence of obesity and type 2 diabetes is increasing dramatically worldwide. Type 2 diabetes arises when pancreatic β-cells fail to secrete sufficient insulin to compensate for peripheral insulin resistance, a condition severely aggravated by obesity (Rhodes 2005; Lazar 2005). Type 2 diabetes is now widely viewed as a manifestation of a broader underlying metabolic disorder called the metabolic syndrome, which is characterized by hyperglycemia, hyperinsulinemia, dyslipidemia, hypertension, visceral obesity, and cardiovascular disease (Reaven 1988). The World Health Organization estimates that the current decade will witness a 46% increase in diabetes incidence worldwide (from 151 million to 221 million), with the vast majority of this increase being due to metabolic syndrome-related type 2 diabetes (Zimmet 2001).

Cellular energy and nutrient sensors determine how cells respond to excessive nutrients, and aberrant nutrient and energy sensing is a contributing factor to metabolic syndrome development (Lindsley 2004; Marshall 2006). AMP-activated protein kinase (AMPK) and mammalian target of rapamycin (mTOR) are two well-studied and evolutionarily conserved cellular energy and nutrient sensors. AMPK is activated in response to intracellular ATP depletion and acts to switch the cellular metabolic program from ATP consumption to ATP production (Hardie 1998). In contrast to AMPK, mTOR is activated by sufficient cellular energy or nutrients, particularly amino acids (Proud 2002). Activation of mTOR stimulates cell growth by increasing protein synthesis through phosphorylation of ribosomal S6 kinase (S6K) and eIF4E binding protein (4E-BP) (Gingras 2001). Decreased AMPK activity and elevated mTOR activity have been linked with obesity, diabetes, and cancer (Winder 1999; Manning 2004; Inoki 2005).

Like AMPK and mTOR, PAS kinase (PASK) is a nutrient-responsive protein kinase conserved from yeast to man. The PAS domain of PASK specifically interacts with the kinase catalytic domain and inactivates the kinase in cis (Rutter 2001). Based on biochemical and genetic data, a model has been proposed wherein a small metabolite activates PASK by directly interacting with the PAS domain and disrupting its interaction with the kinase domain (Amezcua 2002; Rutter 2002). Studies in cultured pancreatic β-cells support a role for PASK in nutrient sensing. Specifically, PASK has been shown to be regulated both post-translationally and at the level of gene expression by glucose (da Silva Xavier 2004).

The in vivo role of PASK in glucose and energy homeostasis was addressed in this study. Using PASK^{-/-} mice (Katschinski 2003), it was demonstrated that PASK deletion results in resistance to the phenotypes caused by a high-fat diet, including obesity, insulin resistance and hepatic triglyceride accumulation. This protection is likely due to increased metabolic rate and energy expenditure in PASK^{-/-} mice independent of the activity of AMPK, mTOR and PGC-1. Increased oxidative metabolism and ATP generation are also observed in cultured cells upon acute PASK knockdown by RNAi. In addition to the previously observed post-translational activation, PASK gene expression is stimulated both in liver and in cultured cells upon refeeding. These cellular effects, which recapitulate effects observed in vivo, support the hypothesis that PASK functions as a cell-autonomous regulator of cellular energy balance.

c) Results

(1) Improved Glucose Tolerance, Increased Insulin Sensitivity and Resistance to Obesity in HFD-Fed PASK^{-/-} Mice

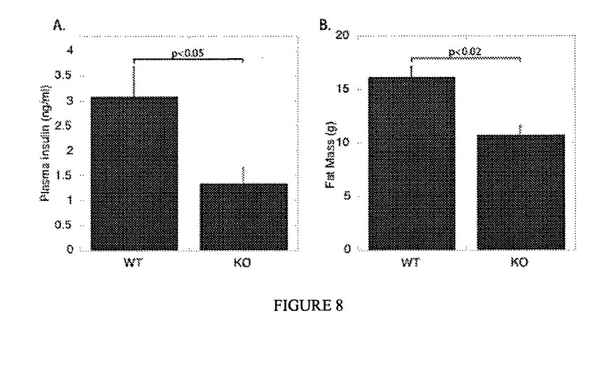

To assess whether PASK is required for the maintenance of glucose homeostasis, a glucose tolerance test (GTT) was performed, in which plasma glucose levels were monitored over time following glucose injection. PASK^{-/-} mice displayed a slight and statistically insignificant glucose intolerance (FIG. 1C). Insulin sensitivity, as measured by insulin tolerance test (ITT), was identical in WT and PASK^{-/-} mice (FIG. 1D). When fed a high-fat diet (HFD), C57BL/6J mice develop obesity and a set of symptoms reminiscent of the metabolic syndrome, including insulin resistance (Surwit 1998). To examine glucose homeostasis under this stress condition, GTT and ITT experiments were performed on HFD-fed WT and PASK^{-/-} mice. While HFD-fed WT mice developed glucose intolerance and insulin resistance, PASK^{-/-} mice were completely protected from these effects (FIGS. 1E, 1F). As expected, fasting insulin levels of HFD-fed PASK^{-/-} mice were substantially lower than that of WT mice (FIG. 8).

PASK^{-/-} mice were also protected from HFD-induced obesity. WT and PASK^{-/-} mice were of similar weight on NCD (FIG. 2A), but the weight gain of PASK^{-/-} mice on HFD was significantly less than that of WT littermates (FIG. 2B). After 8 weeks of HFD-feeding, the body weight of PASK^{-/-} mice was similar to NCD-fed WT or PASK^{-/-} mice. Analysis of body composition by dual energy X-ray absorptiometry showed that the difference in total body weight between WT and PASK^{-/-} HFD-fed mice is completely accounted for by decreased fat mass in PASK^{-/-} mice (FIG. 8).

(2) PASK^{-/-} Mice Exhibit Increased Whole Body Energy Expenditure and Increased Metabolic Rate in Skeletal Muscle

It was hypothesized that the PASK^{-/-} lean phenotype is responsible for the improved glucose tolerance and insulin sensitivity; therefore the basis for this phenotype was sought. Obesity is the result of an imbalance between energy intake (in the form of feeding) and energy expenditure (in the form of physical activity and basal metabolism) (Spiegelman 2001). O₂ consumption, CO₂ production, food intake and locomotor activity were then measured using metabolic chambers. Food intake and locomotor activity were similar, but PASK^{-/-} mice consumed more O₂, produced more CO₂ and generated more heat than WT littermates (FIG. 3A). This hypermetabolic phenotype in PASK^{-/-} mice was also manifest in permeabilized soleus muscle fibers, wherein we observed elevated ATP production from succinate in PASK^{-/-} muscle (FIG. 3B).

One potential explanation for the observed increase in oxidative metabolism was an increase in mitochondrial mass in PASK^{-/-} muscle. However, soleus muscle electron micrographs (FIG. 3C) and subsequent quantification of both mitochondrial number and area showed no difference between PASK^{-/-} and WT soleus muscle (FIG. 3D). Further, the activity of citrate synthase, a marker of mitochondrial density (Leek 2001), was identical in PASK^{-/-} and WT soleus muscle extracts (FIG. 3E). No difference in the mRNA levels of either PGC-1α or PGC-1β were observed, important transcriptional regulators of mitochondrial biogenesis (Leone 2005; Puigserver 1998). It was concluded that PASK deficiency leads to increased mitochondrial metabolism and ATP production that is independent of increased mitochondrial biogenesis.

(3) PASK^{-/-} Mice Exhibit Reduced Liver Triglyceride Accumulation

Diet-induced obesity is typically accompanied by increased lipid accumulation in peripheral tissues, which is strongly associated with the development of insulin resistance (Unger 2002; Voshol 2003). Liver lipid content was examined both by histological Oil-Red-0 staining (FIG. 2C) and by enzymatic triglyceride quantification (FIG. 2D). In both cases, HFD-fed PASK−/− mice were completely protected from the increased lipid accumulation observed in RFD-fed WT mice.

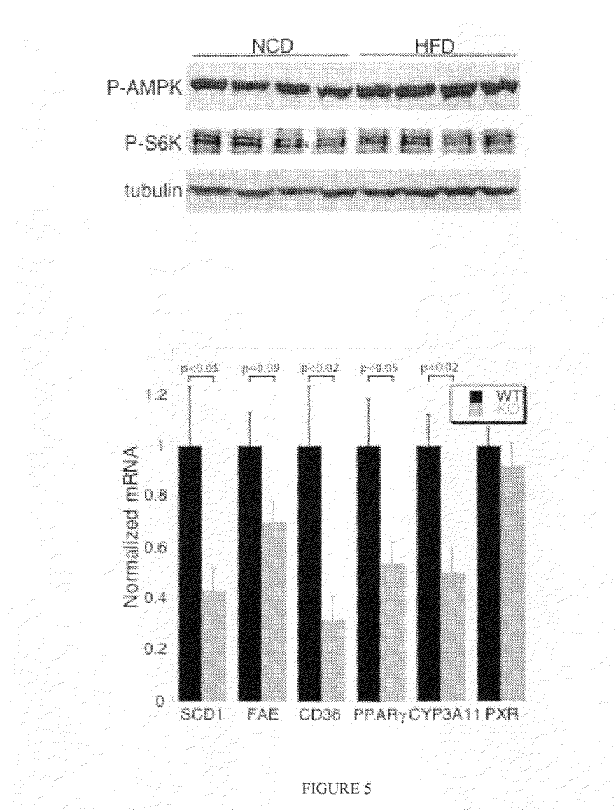

Increased AMPK activity and reduced mTOR pathway activity cause resistance to diet-induced obesity, as demonstrated using transgenic and pharmacologic strategies (Um 2004; Zhou 2001). Given their joint role as nutrient sensors, it was sought whether PASK deletion affected AMPK or mTOR activity. Western blots of WT and PASK^{-/-} liver samples from NCD-fed and HFD-fed mice were preformed using antibodies recognizing phospho-AMPK (Thr172) and phospho-S6K (Thr389), which are widely accepted as markers of AMPK and mTOR activity respectively. As shown in FIG. 5A, we observed no difference among these groups. It was also observed no change in phospho-AMPK and phospho-S6K in gastrocnemius muscle, showing the function of PASK is independent of changes in the activity of the AMPK or mTOR pathways.

It was also determined that the levels of transcripts related to lipid metabolism in liver from HFD-fed WT and HFD-fed PASK^{-/-} mice. Levels of stearoyl-coA desaturase 1 (SCD1) (Flowers 2006), long chain fatty acid elongase (FAE) (Matsuzaka 2002), fatty acid transporter (FAT or CD36) (Schaffer 2002), and the lipid-responsive nuclear hormone receptor PPARγ (Matsusue 2003) are all significantly decreased in PASK^{-/-} liver (FIG. 5B). Lower expression of each of these genes is consistent with decreased hepatic lipid synthesis and triglyceride accumulation. The transcripts of other genes involved in fatty acid metabolism including FAS, ACC-1 and SREBP-1c showed no difference between WT and PASK^{-/-} liver (Table 3).

The altered lipid accumulation and the pattern of gene expression observed in PASK^{-/-} liver is strikingly similar, albeit opposite, to that observed in a transgenic mouse expressing a constitutively active pregnane X receptor (PXR) in liver (Zhou 2006). This raised the possibility that the PASK-dependent effects on lipid metabolism in liver might be mediated through a decrease in PXR expression or function. Indeed, a decrease in the mRNA levels of a well-established PXR target gene CYP3A11 (Goodwin 2002) in PASK^{-/-} liver was observed. The expression of the PXR gene itself is not altered by PASK deletion (FIG. 5B), therefore, if PASK does regulate PXR activity it does so via a mechanism distinct from gene expression.

(4) Acute PASK Silencing Increases Oxidative Metabolism in Cultured Cells

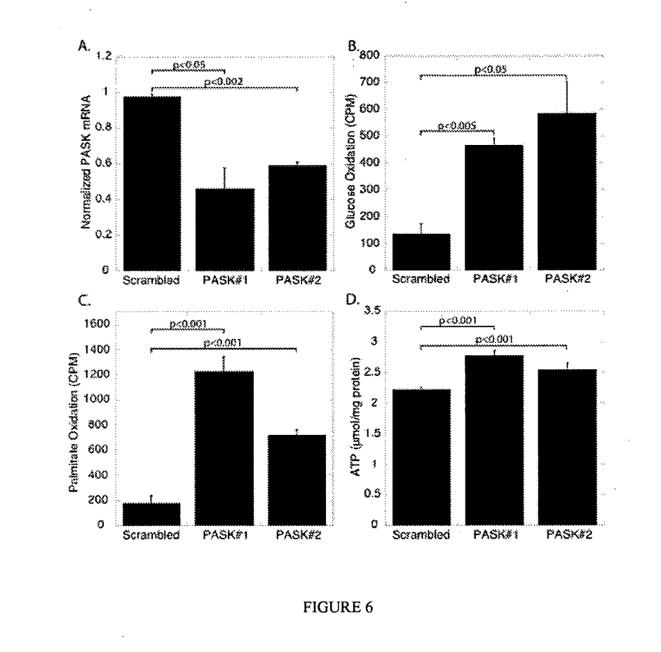

To address the possibility that the observed hypermetabolic phenotype in skeletal muscle is a secondary or adaptive response to PASK deletion in mice, two independent L6 myoblast cell lines were generated wherein PASK expression was acutely silenced using shRNA upon doxycycline removal. As shown in FIG. 6A, these two clones both exhibit an approximately 50% decrease in PASK mRNA upon doxycycline removal compared with the scrambled shRNA control. Upon PASK knockdown, we observed a large increase in both glucose and palmitate oxidation (FIGS. 6B and 6C). This increased substrate metabolism is accompanied by elevated steady state levels of ATP, presumably a result of increased mitochondrial ATP production (FIG. 6D). L6-derived cells with constitutive knockdown of PASK were also generated, and observed increased glucose oxidation and ATP levels. These data in cultured cells indicate that loss of PASK leads to an acute and cell-autonomous increase in mitochondrial metabolism and ATP production.

(5) PASK Expression is Regulated by Feeding Status

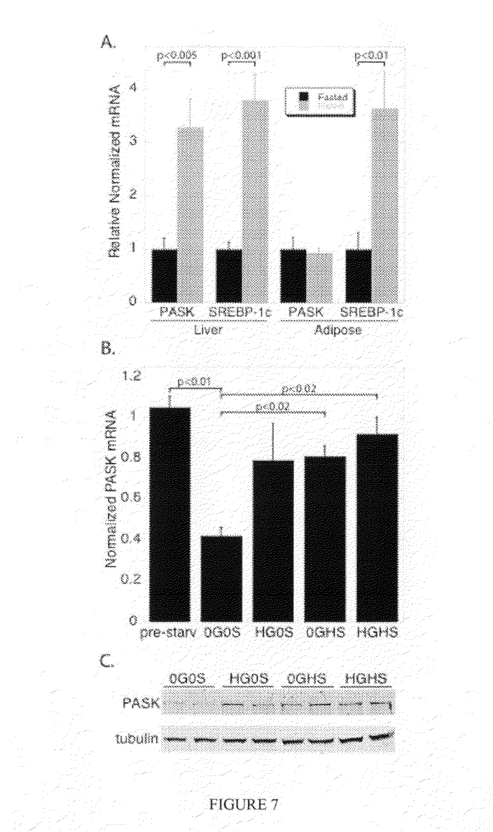

It has been proposed that PASK as an allosteric sensor of cellular metabolic state. To determine if PASK expression is also regulated by metabolic state in vivo, PASK mRNA levels were measured in various tissues from mice fasted for 19 hr and from mice fasted for 12 hr and refed for 7 hr. As shown in FIG. 7A, PASK mRNA levels in liver increase 3-fold upon refeeding relative to fasted levels. PASK mRNA levels in adipose tissue, however, are unchanged by feeding status. SREBP-1c, a known refeeding induced gene (Gosmain 2005), is upregulated 3 to 4-fold in both liver and adipose tissue in these animals. The induction of PASK mRNA in liver was similar in HFD-fed mice and PASK expression was also induced in skeletal muscle by refeeding.

Whether the effect of feeding status on PASK gene expression is a cell-autonomous nutrient sensing response was sought. PASK mRNA levels were measured in human hepatoma HepG2 cell line under different “feeding” regimens. As shown in FIG. 7B, 31 hr starvation in medium lacking glucose and serum reduced PASK mRNA levels roughly 3-fold. Following 24 hr starvation, incubation with either glucose or serum for 7 hr restored PASK mRNA levels to nearly pre-starvation levels, as did incubation with both glucose and serum. PASK protein levels are affected in parallel with PASK mRNA as shown by Western blotting of identically treated cells (FIG. 7C).

d) Discussion

Herein a physiological role for PASK in regulating mammalian energy balance is described. PASK^{-/-} mice are protected from HFD-induced obesity and from other metabolic perturbations that accompany HFD-induced obesity. Insulin sensitivity and glucose tolerance in HFD-fed PASK^{-/-} mice are nearly identical to that of NCD-fed WT or PASK^{-/-} mice. These changes in organismal energy and glucose homeostasis are likely manifestations of an underlying alteration in metabolic regulation in individual cells and tissues.

A striking protection from HFD-induced hepatic steatosis in PASK^{-/-} mice was observed. This is accompanied by a significant decrease in the transcript levels of SCD-1, FAE, CD36 and PPARγ. SCD-1 is the rate-limiting enzyme in the synthesis of mono-unsaturated fatty acids, which are the major substrates for triglyceride synthesis (Dobryzn 2005). SCD-1^{-/-} mutant mice exhibit decreased liver triglyceride accumulation and fatty acid biosynthesis and are protected from HFD-induced obesity (Miyazaki 2000; Ntambi 2002). Fatty acid elongase (FAE) is required for de novo fatty acid synthesis (Jakobsen 2006). CD36 (also known as fatty acid translocase or FAT) is a putative fatty acid transporter. Interestingly, CD36 is a target gene of PPARγ (Tontonoz 1998), thus the decreased expression of CD36 could be secondary to the lower expression of PPARγ in PASK^{-/-} liver. PPARγ expression has been shown to positively correlate with obesity in multiple mouse models (Memon 2000). Decreased expression of each of these genes in liver is consistent with decreased lipid content observed in PASK^{-/-} mice. These alterations in gene expression, and particularly a decrease in CYP3A11 expression, are consistent with decreased PXR activity in PASK^{-/-} liver³¹. The expression of PXR is not different, however, suggesting that PASK might regulate PXR via direct phosphorylation, through altered abundance of a PXR agonist or through other mechanisms. Alternatively, the PXR paralog CAR, which activates an overlapping set of genes, can be down-regulated upon PASK deletion.

Deletion of PASK leads to organismal hypermetabolism as measured by O₂ consumption, CO₂ production and heat generation. This hypermetabolism is also exhibited in isolated permeabilized skeletal muscle, wherein increased ATP production was observed. A number of mouse models exhibit similar hypermetabolism. Three aspects of the PASK^{-/-} phenotype make it unusual. First, the elevated metabolic rate is not caused by impaired metabolic efficiency and concomitantly increased substrate demand. In fact, increased ATP production both in isolated PASK^{-/-} skeletal muscle and in cultured cells upon acute PASK knockdown was observed. Further, energy stress is not apparent, as there is no AMPK hyperactivation in liver or skeletal muscle from PASK^{-/-} mice as measured by either AMPK or ACC phosphorylation (FIG. 5A). Second, the increased mitochondrial oxidative metabolism occurs without changes in mitochondrial mass or number or in PGC-1α, or β expression. Third, this phenotype seems to be a property of individual cells as it is recapitulated in L6 myoblasts upon acute knockdown of PASK mRNA. Increased oxidative metabolism upon PASK loss appears to be due to changes in mitochondria. The ex vivo soleus ATP measurements were performed under conditions that measure mitochondrial ATP production. Further, the observation that both glucose and palmitate oxidation were increased upon PASK knockdown shows that the alteration lies downstream of where these two metabolic pathways converge, namely at the level of the mitochondrial TCA cycle.

PASK is post-translationally activated by elevated glucose medium in cultured β-cells, probably through allosteric control via its regulatory PAS domain. It is now shown that PASK is also activated at the level of gene expression by favorable nutrient conditions both in liver and in cultured cells. Taken together, it is concluded that PASK integrates multiple cues to monitor cellular energetic status. As inferred from the loss of function phenotype, the effect of PASK activation seems to be cell type specific and part of an appropriate nutrient response for each cell type analyzed. PASK activation in hepatocytes increases the synthesis and accumulation of storage lipids such as triglyceride. PASK activation in skeletal muscle results in decreased ATP generation both from carbohydrate and fatty acid oxidation. A model is therefore given wherein PASK acts as a sensor, integrator and transducer of a metabolic sufficiency signal. The transduction pathway downstream of PASK is cell type specific, but at least in skeletal muscle, decreases mitochondrial oxidative metabolism and ATP production. When PASK is artificially lost, this metabolic sufficiency signal is not transduced and the result is chronically elevated mitochondrial metabolism, resulting in protection from ectopic HFD-induced lipid accumulation. As with the other well known metabolic sensory kinases AMPK and mTOR, PASK is an important regulator of human metabolic disease.

e) Methods

Animals. PASK^{-/-} mice were genotyped as described (Katschinski 2003). After the 5th backcross into C57BL/6 (Charles River Laboratories), 12 to 24 week old males were used for all experiments. Mice were maintained on a normal chow diet (Harlan Teklad 3080) or a high fat diet from 12 weeks of age (45% fat by calories, Research Diets, D12451). In each experiment, age-matched wild-type littermates were used as controls for PASK^{-/-} mice.

Cell Culture. Rat L6 myoblasts were provided by Dr. Scott Summers (U. of Utah) and human HepG2 hepatoma cells were from American Type Culture Collection. Both cells were maintained at 37° C. under 5% CO₂ in DMEM supplemented with 10% fetal bovine serum (Hyclone), 0.1 mg/ml penicillin and 0.1 mg/ml streptomycin (Life Technologies).

Glucose/Insulin tolerance test and serum insulin measurement. For GTT and plasma insulin measurement, experimental animals were fasted for 6 hr, after which glucose (1 g/kg body weight) was injected intraperitoneally. At the indicated times, tail vein blood was sampled for glucose determination with a glucometer (Bayer Corp.) or for insulin measurement using the Sensitive Rat Insulin RIA Kit (Linco Research). For ITT, human recombinant insulin (Novo Nordisk, 0.75 U/kg body weight) was injected intraperitoneally to random-fed mice and blood glucose levels were determined at the indicated times.

Metabolic chamber studies. Indirect calorimetry was performed with a four-chamber Oxymax system (Columbus Instruments). Animals were allowed to adapt to the metabolic chamber for 4 hr and then VO₂, VCO₂, heat production, food and water intake, and movement were measured every 15 min for 3 days from individually-housed mice. Averaged data from 6 pm to 6 am is expressed as night values while data from 6 am to 6 pm is expressed as day values.

Mitochondrial respiration experiment. Soleus fibers were separated and then permeabilized by saponin. Maximal (ADP-stimulated) ATP production rate was determined by exposing fibers to 1 mM exogenous ADP and succinate as described (Leone 2005).

Citrate synthase activity assay. Citrate synthase activity was measured by the spectrophotometric method as described (Boudina 2005). Briefly, frozen soleus muscle was homogenized on ice and citrate synthase were released from mitochondria by freezing and thawing the homogenate. The reaction was initiated by addition of oxaloacetate into 1 ml diluted homogenate in reaction buffer containing acetyl-CoA, and then monitored at 412 nm for 3 min with an Ultrospec 3000 spectrophotometer (Amersham).

Histological analysis. For electron microscopy analysis, soleus muscle were fixed in EM fixative, dehydrated in graded ethanol and embedded in Poly Bed plastic resins for sectioning as described²¹. Soleus mitochondrial number were quantified in blinded fashion from electron micrographs at the magnification of 8000× and normalized to Z line number. For Oil Red O staining, frozen liver samples were embedded in OTC reagent (Tissue-Tek) and sectioned at 8 um in cryostat. Cryosections were fixed in formaldehyde steam at 50° C., incubated in 0.5% Oil Red O in isopropyl alcohol and counter-stained with hematoxylin (Sigma). After rinsing in distilled water, liver sections were mounted with permanent aqueous mounting medium Gel/Mount (Biomeda Corp) and photographed using light microscopy at 40× magnification. All images were acquired at the University of Utah Imaging Core Facility.

Liver triglyceride content measurement. Quantitative analysis of liver triglyceride content was performed by saponification of liver in ethanolic KOH as described (Norris 2003). After neutralization with MgCl₂, glycerol produced during hydrolysis was measured by a colorimetric assay using Free Glycerol Reagent and Glycerol Standard Solution (Sigma).

Western blot. Liver lysates were prepared by homogenizing 50-100 mg snap-frozen liver slices in cell lysis buffer (Cell Signaling Technology, Inc) using a Tissue-Tearer rotor. After centrifugation at 14000 rpm for 30 min at 4° C., the supernatants were collected and protein concentrations were determined by Advanced Protein Assay Reagent (Cytoskeleton, Inc). About 50 μg protein from each sample were separated by SDS-PAGE, transferred to a PVDF membrane (Fisher), and blotted with indicated antibodies following the manufacturer's protocol. Phospho-AMPK (T172), Phospho-S6K (Thr389) and tubulin antibodies were purchased from Cell Signaling Technology, Inc. HepG2 cell lysates were prepared by directly adding 200 μl 1×SDS-PAGE loading buffer to cells in 6-well plates, which have been washed with PBS and frozen in liquid nitrogen. After vortexing and boiling, 40 μl of each sample was loaded and immunoblotted as described above using hPASK and tubulin antibodies.

Real-time quantitative RT-PCR. Total RNA was extracted from tissues or cells using RNAStat60 reagent (Tel-Test Inc.) and purified with RNeasy Mini Kit (Qiagen), according to the manufacturer's instructions. First-strand cDNA synthesis was carried out using Superscript III reverse transcriptase (Invitrogen). Real-time PCR was performed on Roche LightCycler using the SYBR Green-based method as described (Cooksey 2004). Melting curve analysis and a mock reverse transcribed control were included to ensure the specificity of the amplicons.

Generation of inducible PASK knockdown cells. pRevTet-Off-IN retroviral vector was purchased from Clontech, and retrovirus was produced according to the manufacturer's instructions using Fugene transfection reagent (Roche Applied Science) and Phoenix-Ampho retroviral packaging cell line (ATCC). L6 cells were infected with the generated Tet-Off retrovirus in the presence of 6 mg/ml polybrene and 48 hr after infection G418 was added to the medium at a final concentration of 200 μg/ml. After 2 weeks of selection, G418-resistant colonies were individually harvested using cloning cylinders (Corning) and screened for inducibility with pRevTRE-Luc (Clontech) virus. shRNA oligonucleotide duplexes containing sequences targeting rat PASK gene and a scrambled shRNA were designed using Invitrogen website software and cloned into tetracycline-regulated retroviral-SIN-TREmiR30-PIG (TMP) vector (OPEN Biosystems). Retrovirus expressing the inducible hairpins were generated and used to infect highly inducible Tet-Off L6 clones as verified by luciferase assay. Finally, 2 μg/ml puromycin as well as 2 μg/ml doxycycline was applied and puromycin-resistant clones were isolated as described above. PASK knockdown clones were screened by qRT-PCR analysis of PASK mRNA levels in the absence and presence of doxycycline. PASK targeting hairpin sequence: GATGCCAAGACCACAGAGA (SEQ ID NO: 1) and GCGCAGACAAGCTCAAAGA (SEQ ID NO: 2). Scrambled sequence: GCGCAGACAAGCTCAAAGA (SEQ ID NO: 3).

Glucose and palmitate oxidation measurement. Glucose oxidation rates were determined according to previously described methods (Antinozzi 1998). Briefly, triplicate samples of L6 cells were incubated with 500 μl oxygenated Krebs-Ringer Buffer containing 5 mM unlabelled glucose, 2 μCi [U-¹⁴C]glucose (MP Biochemicals) and 0.4% BSA (w/v) in a 24-well plate. A UniFilter-24 GF/B plate (Packard Instruments) was sealed with an adhesive sheet using vacuum grease, and 200 μl of 10× hyamine hydroxide (PerkinElmer Sciences) was pipetted onto each filter for CO₂ capture. Finally, the 24-well plate was sealed with a rubber gasket. The apparatus was incubated with gentle shaking for 2 hr at 37° C. and the experiment was stopped by injecting 1000 μM perchloric acid per well. Filters were removed, and captured ¹⁴CO₂ was measured by scintillation counting. Control incubations lacking cells were included in each plate. For palmitate oxidation, the same procedure was used except using KRB buffer containing 1 mM glucose, 0.5 mM unlabelled palmitate, 1 μCi [1-¹⁴C]palmitate (MP Biochemicals) and 1 mM carnitine.

Cellular ATP content measurement. L6 cells were washed with PBS, harvested by trypsinization, and pelleted by centrifugation. The cells were then lysed in 1M perchloric acid on ice to precipitate cellular proteins. After centrifugation at 14000 rpm for 10 min, the supernatant were transferred into a new tube and neutralized with equal volume of 1M KOH. The ATP content was measured by ATP Determination Kit (Invitrogen) according to manufacturer's instructions.

Statistical analysis. Data are presented as average±standard deviation unless otherwise indicated. A two-tailed equal variance t-test was used to compare differences, and the null hypothesis was rejected at the 0.05 level.

TABLE 3
Normalized transcript levels in KO liver (WT set as 1)

| Transcript | KO ave (WT = 1) | St. Dev. | P-value (vs. WT) |
|---|---|---|---|
| Glut2 | 0.92 | 0.17 | 0.37 |
| G6Pase | 0.66 | 0.34 | 0.16 |
| PEPCK | 0.88 | 0.23 | 0.38 |
| PGC-1α | 0.88 | 0.17 | 0.24 |
| PGC-1β | 1.00 | 0.14 | 1.00 |
| PPARα | 0.90 | 0.12 | 0.17 |
| LXRα | 1.02 | 0.13 | 0.82 |
| SREBP-1c | 0.75 | 0.26 | 0.32 |
| FAS | 0.74 | 0.30 | 0.53 |
| ACL | 0.93 | 0.52 | 0.85 |
| MCAD | 0.79 | 0.42 | 0.46 |
| LCAD | 1.00 | 0.14 | 1.00 |
| VLCAD | 1.03 | 0.15 | 0.70 |

### F. REFERENCES

Kahn, B. B., Alquier, T., Carling, D. & Hardie, D. G. AMP-activated protein kinase: ancient energy gauge provides clues to modern understanding of metabolism. Cell Metab 1, 15-25 (2005).
Shaw, R. J. et al. The tumor suppressor LKB1 kinase directly activates AMP-activated kinase and regulates apoptosis in response to energy stress. Proc Natl Acad Sci USA 101, 3329-35 (2004).
Liu, Y., Wan, Q., Guan, Q., Gao, L. & Zhao, J. High-fat diet feeding impairs both the expression and activity of AMPKa in rats' skeletal muscle. Biochem Biophys Res Commun 339, 701-7 (2006).
da Silva Xavier, G. et al. Role for AMP-activated protein kinase in glucose-stimulated insulin secretion and preproinsulin gene expression. Biochem J 371, 761-74 (2003).
Richards, S. K., Parton, L. E., Leclerc, I., Rutter, G. A. & Smith, R. M. Over-expression of AMP-activated protein kinase impairs pancreatic {beta}-cell function in vivo. J Endocrinol 187, 225-35 (2005).
Hardie, D. G. Minireview: the AMP-activated protein kinase cascade: the key sensor of cellular energy status. Endocrinology 144, 5179-83 (2003).
Cool et al. Identification and characterization of a small molecule AMPK activator that treats key components of type 2 diabetes and the metabolic syndrome. Cell Metabolism 3, 406-416 (2006).
Rhodes, C. J. Type 2 diabetes—a matter of beta-cell life and death? Science. 307, 380-4. (2005).
Lazar, M. A. How obesity causes diabetes: not a tall tale. Science. 307, 373-5. (2005).
Reaven, G. M. Banting lecture 1988. Role of insulin resistance in human disease. Diabetes. 37, 1595-607. (1988).
Zimmet, P., Alberti, K. G. & Shaw, J. Global and societal implications of the diabetes epidemic. Nature. 414, 782-7. (2001).
Lindsley, J. E. & Rutter, J. Nutrient sensing and metabolic decisions. Comp Biochem Physiol B Biochem Mol Biol 139, 543-59. (2004).
Marshall, S. Role of insulin, adipocyte hormones, and nutrient-sensing pathways in regulating fuel metabolism and energy homeostasis: a nutritional perspective of diabetes, obesity, and cancer. Sci STKE. 2006, re7. (2006).
Hardie, D. G., Carling, D. & Carlson, M. The AMP-activated/SNF1 protein kinase subfamily: metabolic sensors of the eukaryotic cell? Annual Review of Biochemistry 67, 821-55 (1998).
Proud, C. G. Regulation of mammalian translation factors by nutrients. Eur J Biochem 269, 5338-49. (2002). Gingras, A. C., Raught, B. & Sonenberg, N. Regulation of translation initiation by FRAP/mTOR. Genes Dev. 15, 807-26. (2001).
Winder, W. W. & Hardie, D. G. AMP-activated protein kinase, a metabolic master switch: possible roles in type 2 diabetes. Am J Physiol. 277, E1-10. (1999).
Manning, B. D. Balancing Akt with S6K: implications for both metabolic diseases and tumorigenesis. J Cell Biol. 167, 399-403. (2004).
Inoki, K., Corradetti, M. N. & Guan, K. L. Dysregulation of the TSC-mTOR pathway in human disease. Nat Genet. 37, 19-24. (2005).
Rutter, J., Michnoff, C. H., Harper, S. M., Gardner, K. H. & McKnight, S. L. PAS kinase: An evolutionarily conserved PAS domain-regulated serine/threonine kinase. Proceedings of the National Academy of Sciences of the United States of America 98, 8991-8996 (2001).
Amezcua, C. A., Harper, S. M., Rutter, J. & Gardner, K. H. Structure and interactions of PAS kinase N-terminal PAS domain: model for intramolecular kinase regulation. Structure (Camb) 10, 1349-61. (2002).
Rutter, J. Essay: Amersham Biosciences and Science Prize. PAS domains and metabolic status signaling. Science 298, 1567-8. (2002).
da Silva Xavier, G., Rutter, J. & Rutter, G. A. Involvement of Per-Amt-Sim (PAS) kinase in the stimulation of preproinsulin and pancreatic duodenum homeobox 1 gene expression by glucose. Proc Natl Acad Sci USA 101, 8319-24. Epub 2004 May 17. (2004).
Katschinski, D. M. et al. Targeted disruption of the mouse PAS domain serine/threonine kinase PASKIN. Mol Cell Biol 23, 6780-9 (2003).
Surwit, R. S., Kuhn, C. M., Cochrane, C., McCubbin, J. A. & Feinglos, M. N. Diet-induced type II diabetes in C57BL/6J mice. Diabetes. 37, 1163-7. (1988).
Spiegelman, B. M. & Flier, J. S. Obesity and the regulation of energy balance. Cell. 104, 531-43. (2001).
Leek, B. T., Mudaliar, S. R., Henry, R., Mathieu-Costello, O. & Richardson, R. S. Effect of acute exercise on citrate synthase activity in untrained and trained human skeletal muscle. Am J Physiol Regul Integr Comp Physiol. 280, R441-7. (2001).
Leone, T. C. et al. PGC-1alpha deficiency causes multi-system energy metabolic derangements: muscle dysfunction, abnormal weight control and hepatic steatosis. PLoS Biol. 3, e101. Epub 2005 Mar. 15. (2005).
Puigserver, P. et al. A cold-inducible coactivator of nuclear receptors linked to adaptive thermogenesis. Cell. 92, 829-39. (1998).
Unger, R. H. Lipotoxic diseases. Annu Rev Med. 53, 319-36. (2002).
Voshol, P. J. et al. Increased hepatic insulin sensitivity together with decreased hepatic triglyceride stores in hormone-sensitive lipase-deficient mice. Endocrinology. 144, 3456-62. (2003).
Um, S. H. et al. Absence of S6K1 protects against age- and diet-induced obesity while enhancing insulin sensitivity. Nature. 431, 200-5. Epub 2004 Aug. 11. (2004).
Zhou, G. et al. Role of AMP-activated protein kinase in mechanism of metformin action. J Clin Invest 108, 1167-74. (2001).
Flowers, M. T., Miyazaki, M., Liu, X. & Ntambi, J. M. Probing the role of stearoyl-CoA desaturase-1 in hepatic insulin resistance. J Clin Invest. 116, 1478-81. (2006).
Matsuzaka, T. et al. Cloning and characterization of a mammalian fatty acyl-CoA elongase as a lipogenic enzyme regulated by SREBPs. J Lipid Res. 43, 911-20. (2002).
Schaffer, J. E. Fatty acid transport: the roads taken. Am J Physiol Endocrinol Metab. 282, E239-46. (2002).
Matsusue, K. et al. Liver-specific disruption of PPARgamma in leptin-deficient mice improves fatty liver but aggravates diabetic phenotypes. J Clin Invest. 111, 737-47. (2003).
Zhou, J. et al. A novel pregnane X receptor-mediated and sterol regulatory element-binding protein-independent lipogenic pathway. J Biol Chem. 281, 15013-20. Epub 2006 Mar. 23. (2006).
Goodwin, B., Redinbo, M. R. & Kliewer, S. A. Regulation of cyp3a gene transcription by the pregnane x receptor. Annu Rev Pharmacol Toxicol. 42, 1-23. (2002).
Gosmain, Y. et al. Regulation of SREBP-1 expression and transcriptional action on HKII and FAS genes during fasting and refeeding in rat tissues. J Lipid Res. 46, 697-705. Epub 2005 Jan. 1. (2005).
Dobrzyn, A. & Ntambi, J. M. Stearoyl-CoA desaturase as a new drug target for obesity treatment. Obes Rev. 6, 169-74. (2005).
Miyazaki, M., Kim, Y. C., Gray-Keller, M. P., Attie, A. D. & Ntambi, J. M. The biosynthesis of hepatic cholesterol esters and triglycerides is impaired in mice with a disruption of the gene for stearoyl-CoA desaturase 1. J Biol Chem. 275, 30132-8. (2000).
Ntambi, J. M. et al. Loss of stearoyl-CoA desaturase-1 function protects mice against adiposity. Proc Natl Acad Sci USA. 99, 11482-6. Epub 2002 Aug. 12. (2002).
Jakobsson, A., Westerberg, R. & Jacobsson, A. Fatty acid elongases in mammals: their regulation and roles in metabolism. Prog Lipid Res. 45, 237-49. Epub 2006 Mar. 6. (2006).
Tontonoz, P., Nagy, L., Alvarez, J. G., Thomazy, V. A. & Evans, R. M. PPARgamma promotes monocyte/macrophage differentiation and uptake of oxidized LDL. Cell. 93, 241-52. (1998).
Memon, R. A. et al. Up-regulation of peroxisome proliferator-activated receptors (PPAR-alpha) and PPAR-gamma messenger ribonucleic acid expression in the liver in murine obesity: troglitazone induces expression of PPAR-gamma-responsive adipose tissue-specific genes in the liver of obese diabetic mice. Endocrinology. 141, 4021-31. (2000).
Boudina, S. et al. Reduced mitochondrial oxidative capacity and increased mitochondrial uncoupling impair myocardial energetics in obesity. Circulation. 112, 2686-95. (2005).
Norris, A. W. et al. Muscle-specific PPARgamma-deficient mice develop increased adiposity and insulin resistance but respond to thiazolidinediones. J Clin Invest. 112, 608-18. (2003).
Cooksey, R. C. et al. Oxidative stress, beta-cell apoptosis, and decreased insulin secretory capacity in mouse models of hemochromatosis. Endocrinology. 145, 5305-12. Epub 2004 Aug. 12. (2004).
Antinozzi, P. A., Segall, L., Prentki, M., McGarry, J. D. & Newgard, C. B. Molecular or pharmacologic perturbation of the link between glucose and lipid metabolism is without effect on glucose-stimulated insulin secretion. A re-evaluation of the long-chain acyl-CoA hypothesis. J Biol Chem. 273, 16146-54. (1998).

### G. SEQUENCES

SEQ ID NO: 1

PASK#1
GATGCCAAGACCACAGAGA

SEQ ID NO: 2

PASK#2
GCGCAGACAAGCTCAAAGA

SEQ ID NO: 3

Scrambled
GCGCAGACAAGCTCAAAGA

SEQ ID NO: 4

Human PASK
MEDGGLTAFEEDQRCLSQSLPLPVSAEGPAAQTTAEPSRSFSSAHRHLSR
RNGLSRLCQSRTALSEDRWSSYCLSSLAAQNICTSKLHCPAAPEHTDPSE
PRGSVSCCSLLRGLSSGWSSPLLPAPVCNPNKAIFTVDAKTTEILVANDK
ACGLLGYSSQDLIGQKLTQFFLRSDSDVVEALSEEHMEADGHAAVVFGTV
VDIISRSGEKIPVSVWMKRMRQERRLCCVVVLEPVERVSTWVAFQSDGTV
TSCDSLFAHLHGYVSGEDVAGQHITDLIPSVQLPPSGQHIPKNLKIQRSV
GRARDGTTFPLSLKLKSQPSSEEATTGEAAPVSGYRASVWVFCTISGLIT
LLPDGTIHGINHSFALTLFGYGKTELLGKNITFLIPGFYSYMDLAYNSSL
QLPDLASCLDVGNESGCGERTLDPWQGQDPAEGGQDPRINVVLAGGHVVP
RDEIRKLMESQDIFTGTQTELIAGGQLLSCLSPQPAPGVDNVPEGSLPVH
GEQALPKDQQITALGREEPVAIESPGQDLLGESRSEPVDVKPFASCEDSE
APVPAEDGGSDAGMCGLCQKAQLERMGVSGPSGSDLWAGAAVAKPQAKGQ
LAGGSLLMHCPCYGSEWGLWWRSQDLAPSPSGMAGLSFGTPTLDEPWLGV
ENDREELQTCLIKEQLSQLSLAGALDVPHAELVPTECQAVTAPVSSCDLG
GRDLCGGCTGSSSACYALATDLPGGLEAVEAQEVDVNSFSWNLKELFFSD
QTDQTSSNCSCATSELRETPSSLAVGSDPDVGSLQEQGSCVLDDRELLLL
TGTCVDLGQGRRFRESCVGHDPTEPLEVCLVSSEHYAASDRESPGHVPST
LDAGPEDTCPSAEEPRLNVQVTSTPVIVMRGAAGLQREIQEGAYSGSCHH
RDGLRLSIQFEVRRVELQGPTPLFCCWLVKDLLHSQRDSAARTRLFLASL
PGSTHSTAAELTGPSLVEVLRARPWFEEPPKAVELEGLAACEGEYSQKYS
TMSPLGSGAFGFVWTAVDKEKNKEVVVKFTKKEKVLEDCWIEDPKLGKVT
LEIAILSRVEHANIIKVLDIFENQGFFQLVMEKHGSGLDLFAFIDRHPRL
DEPLASYIFRQLVSAVGYLRLKDIIHRDIKDENIVIAEDFTIKLIDFGSA
AYLERGKLFYTFCGTIEYCAPEVLMGNPYRGPELEMWSLGVTLYTLVFEE
NPFCELEETVEAAIHPPYLVSKELMSLVSGLLQPVPERRTTLEKLVTDPW
VTQPVNLADYTWEEVCRVNKPESGVLSAASLEMGNRSLSDVAQAQELCGG
PVPGEAPNGQGCLHPGDPRLLTS

SEQ ID NO: 5

Human PASK cDNA
GCCGGCTTGGCGTGACCCTCGCCTGATCCAGTTGTTAGAGTTGGAAGCTT
GGCAGTTGGCCTCCCTTCTTCCCATGGAGGACGGGGGCTTAACAGCCTTT
GAAGAGGACCAGAGATGCCTTTCCCAGAGCCTCCCCTTGCCAGTGTCAGC
AGAGGGCCCAGCTGCACAGACCACTGCTGAGCCCAGCAGGTCGTTTTCCT
CAGCCCACAGACACCTGAGCAGAAGGAATGGGCTTTCCAGACTCTGCCAG
AGCAGGACaGCGCTCTCTgAAGACAGATGGAGCTCCTATTGTCTATCATC
ACTGGCTGCCCAGAATATTTGTACAAGTAAACTGCACTgccctgctgccc
ctgagcacacggacccgtccgaaccgcggggcagtgtgtcctgctgctcc
ctgctgcggggactgtcctcagggtggtcctcacctctgcttccggcccc
tgtgtgcaaccctaacaaggccatcttcacggtggatgccaagaccacag
agatcctGgttgctaacgacaaagcttgcgggctcctggggtacagcagc
caggacctgattggccagaagctcacgcagttctttctgaggtcagattc
tgatgtggtggaggccctcagcgaggagcacatggaggccgacggccacg
ctgcggtggtgtttggcacggtggtggacatcatcaGccgtagtggggag
aagattcccagtgtctgtgtggatgaagaggatgcggcaggagcgccgcc
tatgctgcgtggtggtcctggagcccgtggagagggtctcgacctgggtc
gctttccagagcgatggcaccgtcacgtcatgtgacagtctctttgctca
tcttcacgggtacgtgtctggggaggacgtggctgggcagcatatcacag
acctgatcccttctgtgcagctccctccttctggccagcacatcccaaag
aatctcaagattcagaggtctgttggaagagccagggacggtaccacctt
ccctctgagcttaaagctgaaatcccaacccagcagcgaggaggcgacca
ccggtgaggcggcccctgtgagcggctaccgggcatctgtctgggtgttc
tgcaccatcagtggcctcatcaccctcctgccggatgggaccatccacgg
catcaaccacagcttcgcgctgacactgtttggttacggaaagacggagc
tcctgggcaagaatatcactttcctgattcctggtttctacagctacatg
gaccttgcgtacaacagctcattacagctcccagacctggccagctgcct
ggacgtcggcaatgagagtgggtgtggggagagaaccttggacccgtggc
agggccaggacccagctgaggggggccaggatccaaggattaatgtcgtg
cttgctggtggccacgttgtgccccgagatgagatccggaagctgatgga
aagccaagacatcttcaccgggactcagactgagctgattgctggaggcc
agctcctttcctgcctctcacctcagcctgctccaggggtggacaatgtc
ccagaaggaagcctgccagtgcacggtgaacaggcgctgcccaaggacca
gcaaatcactgccttggggagagaggaacctgtggcaatagagagccccg
gacaggatcttctgggagaaagcaggtctgaaccagtggatgtgaagcca
tttgcttcctgcgaagattctgaagctccagtcccagctgaggatggggg
cagtgatgctggcatgtgtggcctgtgtcagaaggcccagctagagcgga
tgggagtcagtggtcccagcggttcagacctttgggctggggctgccgtg
gccaagccccaggccaagggtcagctggcggggggcagcctcctgatgca
ctgcccttgctatgggagtgaatggggcttgtggtggcgaagCcaggact
tggcccccagcccctctgggatggcaggcctctcgtttgggacacctact
ctagatgagccgtggctgggagtggaaaacgaccgagaagagctgcagac
ctgcttgattaaggagcagctgtcccagttgagccttgcAggagccctgg
atgtcccccacgccgaactcgttccgacagagtgccaggctgtcaccgct
cctGtgtcGtcctgcgatctgggaggcagagacctgtgcggtggctgcac
gggcagctcctcagcctgctatgccttggccacggacctccctgggggcc
tggaagcagtggaggcccaggaggttgatgtgaattcgttttcctggaac
ctcaaggaactctttttcagtgaccagacagaccaaacgtcatcaaattg
ttcctgtgctacgtctgaactcagagagacaccctcttccttggcagtgg
gctccgatccagatgtaggcagtctccaggaacaggggtcgtgtgtcctg
gatgacagggagctgttactactgaccggcacctgtgttgaccttggcca
aggccgacggttccgggagagctgtgtgggacatgatccaacagaaccgc
ttgaggtttgtttggtgtcctctgagcattatgcagcaagcgacagagaa
agcccGggacacgttccttccaCgttggatgctggccctgaggacacgtg
cccatcagcagaggagccaaggctgaacgtccaggtcacctccacgcccg
tgatcgtgatgcgcggggctgctggcctgcagcgggagatccaggagggt
gcctactccgggagctgcCaccatcgagaTggcttacggctgagtataca
gtttgaggtgaggcgggtggagctccagggccccacacctctgttctgct
gctggctggtgaaagacctcctccacagccaacgcgactcagccgccagg
acccgcctgttccttgccagcctgcccggctccacccactctaccgctgc
tgagctcaccggacccagcctggtggaagtgctcagagccagaccctggt
ttgaggagccccccaaggctgtggaactggaggggttggcggcctgtgag
ggcgagtactcccaaaagtacagtaccatgagcccgctgggcagtggggc
cttcggcttcgtgtggactgctgtggacaaAgaaaaaaacaaggaggtgg
tggtgaagtttattaagaaggagaaggtcttggaggattgttggattgag
gatcccaaacttgggaaagttactttagagatcgcaattctatccagggt
ggagcacgccaatatcatcaaggtattggatatatttgaaaaccaagggt
tcttccagcttgtgatggagaagcacggctccggcctagacctcttcgct
ttcatcgaccgccaccccaggctggatgagcccctggcgagctacatctt
ccgacaactagtgtcagcagtgggatacctgcgcttgaaggacatcatcc
accgtgacatcaaggatgagaacatcgtgatcgctgaggacttcacaatc
aagctgatagactttggctcggccgcctacttggaaaggggaaaattatt
ttatactttttgtgggaccatcgagtactgtgcaccggaagttctcatgg
ggaatccctacagagggccggagctggagatgtggtctctgggagtcact
ctgtacacgctggtctttgaggagaaccccttctgtgagctggaggagac
cgtggaggctgccatacacccgccatacctggtgtccaaagaactcatga
gccttgtgtctgggctgctgcagccagtccctgagagacgcaccaccttg
gagaagctggtgacagacccgtgggtaacacagcctgtgaatcttgctga
ctatacatgggaagaggtgtgtcgagtaaacaagccagaaagtggagttc
tgtccgctgcgagcctggagatggggaacaggagcctgagtgatgtggcc
caggctcaggagctttgtgggggccccgttccaggcgaggctcctaatgg
ccaaggctgtttgcatcccggggatccccgtctgctgaccagctaaacac
caatttcttcctgcttttctccacttggtttggaaaatcacacagttttc
aggctccatctgtttggagaaaatacattctgaagcatccccaattcacc
ttctaaaaactcatgtgcaggtttgataaacaccagaacagaagacagtg
atgctgGattattttagatttattacatagatttggaattcacttttttc
atgacctagaaaaaaacattccagtgttcaactgttttatattattaaag
ggcttttaatttgtgaacttctgaaggcatgagtgttttctctttctact
tttgtatatgtgcatgttttgtttcctctgacttggtatatgctcatctg
agtgacggatatgtgaaatttgtagaactggttagtcaaatggccagact
atttcattaatttatttcctcaaatgcttttcaaattaaagcacctttgt
tagtaaacagttaaaaaaaaaaaaaaaaaaaaaaaaaaaaaaa

What is claimed is:

1. A method of treating insulin resistance in a subject comprising:
a. selecting a subject in need of treatment for insulin resistance;
b. administering to the subject an effective amount of a composition that inhibits PASK.

2. The method of claim 1, wherein the subject has metabolic syndrome.

3. The method of claim 2, wherein the subject is obese.

4. The method of claim 2, wherein the subject has diabetes.

5. A method of increasing mitochondrial metabolism a cell, comprising inhibiting PASK, thereby increasing mitochondrial metabolism.

6. A method of screening for a test compound that modulates PASK comprising:
a. contacting PASK with a test compound; and
b. detecting interaction between PASK and the test compound; wherein interaction between the test compound and PASK indicates a compound that modulates PASK.

7. The method of claim 6 wherein a plurality of test compounds are contacted with PASK in a high throughput assay system.

8. The method of claim 6 wherein the high throughput assay system comprises an immobilized array of test compounds.

9. The method of claim 6, wherein the high throughput assay system comprises an immobilized array of PASK molecules.

10. A compound identified by the method of claim 6.

11. A method of screening for a test compound that modulates PASK comprising:
a. contacting a transgenic animal that is PASK deficient with a test compound; and
b. detecting a difference in PASK levels in the transgenic animal; wherein a difference in PASK levels indicates a test compound that modulates PASK.

12. A compound identified by the method of claim 11.

13. A method of treating cancer in a subject, comprising
a. selecting a subject with cancer;
b. administering to the subject an effective amount of a composition that inhibits PASK; thereby treating cancer in the subject.

14. A method of treating diabetes type I in a subject, comprising
a. selecting a subject with diabetes type I; and
b. administering to the subject an effective amount of a composition that inhibits PASK; thereby treating diabetes type I in the subject.

15. A method of treating insulin resistance in a subject comprising:
a. selecting a subject in need of treatment for insulin resistance;
b. administering to the subject a nucleic acid encoding a composition that inhibits PASK.


SEQUENCE LISTING (5 sequences)

SEQ ID NO: 1 - DNA, 19 bp, Artificial Sequence
  Description of Artificial Sequence; note = synthetic construct
gatgccaagaccacagaga

SEQ ID NO: 2 - DNA, 19 bp, Artificial Sequence
  Description of Artificial Sequence; note = synthetic construct
gcgcagacaagctcaaaga

SEQ ID NO: 3 - DNA, 19 bp, Artificial Sequence
  Description of Artificial Sequence; note = synthetic construct
gcgcagacaagctcaaaga

SEQ ID NO: 4 - protein, 1323 aa, Artificial Sequence [sequence omitted]

SEQ ID NO: 5 - DNA, 4542 bp, Artificial Sequence
  Description of Artificial Sequence; note = synthetic construct
gccggcttggcgtgaccctcgcctgatccagttgttagagttggaagcttggcagttggcctcccttcttcccatggaggacgggggcttaacagcctttgaagaggaccagagatgcctttcccagagcctccccttgccagtgtcagcagagggcccagctgcacagaccactgctgagcccagcaggtcgttttcctcagcccacagacacctgagcagaaggaatgggctttccagactctgccagagcaggacagcgctctctgaagacagatggagctcctattgtctatcatcactggctgcccagaatatttgtacaagtaaactgcactgccctgctgcccctgagcacacggacccgtccgaaccgcggggcagtgtgtcctgctgctccctgctgcggggactgtcctcagggtggtcctcacctctgcttccggcccctgtgtgcaaccctaacaaggccatcttcacggtggatgccaagaccacagagatcctggttgctaacgacaaagcttgcgggctcctggggtacagcagccaggacctgattggccagaagctcacgcagttctttctgaggtcagattctgatgtggtggaggccctcagcgaggagcacatggaggccgacggccacgctgcggtggtgtttggcacggtggtggacatcatcagccgtagtggggagaagattccagtgtctgtgtggatgaagaggatgcggcaggagcgccgcctatgctgcgtggtggtcctggagcccgtggagagggtctcgacctgggtcgctttccagagcgatggcaccgtcacgtcatgtgacagtctctttgctcatcttcacgggtacgtgtctggggaggacgtggctgggcagcatatcacagacctgatcccttctgtgcagctccctccttctggccagcacatcccaaagaatctcaagattcagaggtctgttggaagagccagggacggtaccaccttccctctgagcttaaagctgaaatcccaacccagcagcgaggaggcgaccaccggtgaggcggcccctgtgagcggctaccgggcatctgtctgggtgttctgcaccatcagtggcctcatcaccctcctgccggatgggaccatccacggcatcaaccacagcttcgcgctgacactgtttggttacggaaagacggagctcctgggcaagaatatcactttcctgattcctggtttctacagctacatggaccttgcgtacaacagctcattacagctcccagacctggccagctgcctggacgtcggcaatgagagtgggtgtggggagagaaccttggacccgtggcagggccaggacccagctgaggggggccaggatccaaggattaatgtcgtgcttgctggtggccacgttgtgccccgagatgagatccggaagctgatggaaagccaagacatcttcaccgggactcagactgagctgattgctggaggccagctcctttcctgcctctcacctcagcctgctccaggggtggacaatgtcccagaaggaagcctgccagtgcacggtgaacaggcgctgcccaaggaccagcaaatcactgccttggggagagaggaacctgtggcaatagagagccccggacaggatcttctgggagaaagcaggtctgaaccagtggatgtgaagccatttgcttcctgcgaagattctgaagctccagtcccagctgaggatgggggcagtgatgctggcatgtgtggcctgtgtcagaaggcccagctagagcggatgggagtcagtggtcccagcggttcagacctttgggctggggctgccgtggccaagccccaggccaagggtcagctggcggggggcagcctcctgatgcactgcccttgctatgggagtgaatggggcttgtggtggcgaagccaggacttggcccccagcccctctgggatggcaggcctctcgtttgggacacctactctagatgagccgtggctgggagtggaaaacgaccgagaagagctgcagacctgcttgattaaggagcagctgtcccagttgagccttgcaggagccctggatgtcccccacgccgaactcgttccgacagagtgccaggctgtcaccgctcctgtgtcgtcctgcgatctgggaggcagagacctgtgcggtggctgcacgggcagctcctcagcctgctatgccttggccacggacctccctgggggcctggaagcagtggaggcccaggaggttgatgtgaattcgttttcctggaacctcaaggaactctttttcagtgaccagacagaccaaacgtcatcaaattgttcctgtgctacgtctgaactcagagagacaccctcttccttggcagtgggctccgatccagatgtaggcagtctccaggaacaggggtcgtgtgtcctggatgacagggagctgttactactgaccggcacctgtgttgaccttggccaaggccgacggttccgggagagctgtgtgggacatgatccaacagaaccgcttgaggtttgtttggtgtcctctgagcattatgcagcaagcgacagagaaagcccgggacacgttccttccacgttggatgctggccctgaggacacgtgcccatcagcagaggagccaaggctgaacgtccaggtcacctccacgcccgtgatcgtgatgcgcggggctgctggcctgcagcgggagatccaggagggtgcctactccgggagctgccaccatcgagatggcttacggctgagtatacagtttgaggtgaggcgggtggagctccagggccccacacctctgttctgctgctggctggtgaaagacctcctccacagccaacgcgactcagccgccaggacccgcctgttccttgccagcctgcccggctccacccactctaccgctgctgagctcaccggacccagcctggtggaagtgctcagagccagaccctggtttgaggagccccccaaggctgtggaactggaggggttggcggcctgtgagggcgagtactcccaaaagtacagtaccatgagcccgctgggcagtggggccttcggcttcgtgtggactgctgtggacaaagaaaaaaacaaggaggtggtggtgaagtttattaagaaggagaaggtcttggaggattgttggattgaggatcccaaacttgggaaagttactttagagatcgcaattctatccagggtggagcacgccaatatcatcaaggtattggatatatttgaaaaccaagggttcttccagcttgtgatggagaagcacggctccggcctagacctcttcgctttcatcgaccgccaccccaggctggatgagcccctggcgagctacatcttccgacaactagtgtcagcagtgggatacctgcgcttgaaggacatcatccaccgtgacatcaaggatgagaacatcgtgatcgctgaggacttcacaatcaagctgatagactttggctcggccgcctacttggaaaggggaaaattattttatactttttgtgggaccatcgagtactgtgcaccggaagttctcatggggaatccctacagagggccggagctggagatgtggtctctgggagtcactctgtacacgctggtctttgaggagaaccccttctgtgagctggaggagaccgtggaggctgccatacacccgccatacctggtgtccaaagaactcatgagccttgtgtctgggctgctgcagccagtccctgagagacgcaccaccttggagaagctggtgacagacccgtgggtaacacagcctgtgaatcttgctgactatacatgggaagaggtgtgtcgagtaaacaagccagaaagtggagttctgtccgctgcgagcctggagatggggaacaggagcctgagtgatgtggcccaggctcaggagctttgtgggggccccgttccaggcgaggctcctaatggccaaggctgtttgcatcccggggatccccgtctgctgaccagctaaacaccaatttcttcctgcttttctccacttggtttggaaaatcacacagttttcaggctccatctgtttggagaaaatacattctgaagcatccccaattcaccttctaaaaactcatgtgcaggtttgataaacaccagaacagaagacagtgatgctggattattttagatttattacatagatttggaattcacttttttcatgacctagaaaaaaacattccagtgttcaactgttttatattattaaagggcttttaatttgtgaacttctgaaggcatgagtgttttctctttctacttttgtatatgtgcatgttttgtttcctctgacttggtatatgctcatctgagtgacggatatgtgaaatttgtagaactggttagtcaaatggccagactatttcattaatttatttcctcaaatgcttttcaaattaaagcacctttgttagtaaacagttaaaaaaaaaaaaaaaaaaaaaaaaaaaaaaa